(19) United States
(12) Patent Application Publication
(10) Pub. No.: US 2011/0229883 A1
(43) Pub. Date: Sep. 22, 2011

(54) Biochemical Markers for Disease States and Genes for Identification of Biochemical Defects

(76) Inventors: Bernd Spur, Marlton, NJ (US); Ana Rodriguez, Marlton, NJ (US); Peter T. Stein, Cherry Hill, NJ (US); George H. Lambert, Belle Mead, NJ (US); Sue X. Ming, Morganville, NJ (US); William G, Johnson, Short Hills, NJ (US); Edward S. Stenroos, New Brunswick, NJ (US)

(21) Appl. No.: 12/735,900

(22) PCT Filed: Apr. 9, 2008
(86) PCT No.: PCT/US08/04655

§ 371 Date: Jun. 9, 2011

Related U.S. Application Data
(60) Provisional application No. 60/922,699, filed on Apr. 10, 2007.

(51) Int. Cl. C12Q 1/68

(52) U.S. Cl. 435/6.11

(57) ABSTRACT

The present invention relates to a system utilizing biochemical markers and genetic markers to diagnose, predict, and/or monitor intervention of a number of diseases and conditions that have unresolved oxidative stress as an important component. The present invention relates generally to markers and assays for diagnosing, predicting, and monitoring disease, particularly disease-relevant oxidative stress and lipid metabolites and mediators. The oxidative stress, lipid metabolite and lipid mediator biochemical and genetic markers may be further combined with other disease associated or disease relevant markers in methods and assays for diagnosis, monitoring, and assessment of disease, particularly of complex diseases with multi-component factors. The system, methods and assays are applicable to various diseases, including autism, asthma, and Alzheimer's disease.

### CROSS REFERENCE TO RELATED APPLICATIONS

The present application claims priority of co-pending provisional application U.S. Ser. No. 60/922,699, filed on Apr. 9, 2007, the disclosure of which is incorporated by reference herein in its entirety. Applicants claim the benefits of such application under 35 U.S.C. §119(e).

### FIELD OF THE INVENTION

This invention relates to a system utilizing biochemical markers and genetic markers to diagnose, predict, and/or monitor intervention of a number of diseases and conditions that have unresolved oxidative stress as an important component. The present invention relates generally to markers and assays for diagnosing, predicting, and monitoring disease, particularly disease-relevant oxidative stress and lipid metabolites and mediators.

### BACKGROUND OF THE INVENTION

Complex diseases are caused by a combination of multiple genetic and environmental components. The genetic components consist of common variations of small effect. Combinations of these variations interacting with environmental factors will alter the control (ex: induction, regulation) or metabolism (ex: ability to synthesize or degrade) of the biochemical markers. There are multiple signaling cascades and multiple metabolic pathways relevant to the control/metabolism of these biochemical markers.

DNA is vulnerable to oxidative damage and therefore extensive repair mechanisms are present in the cell to minimize damage to DNA and repair any damage that does occur. However the repair processes are not 100% efficient and therefore damaged nucleosides accumulate with age in both nuclear and mitochondrial DNA. The products from the oxidative damage of the four DNA bases are not reincorporated into DNA during DNA repair processes, rather they are excreted into the urine without further metabolism (Shigenaga et al., 1989; Loft and Poulsen, 1998). The most abundant of these oxidized nucleosides, 8-hydroxydeoyguanosine is excreted quantitatively in the urine and as such it has been shown to be a marker for DNA damage (Shigenaga et al., 1989; Loft et al., 1995). Increases in 8-hydroxydeoyguanosine excretion correlate with a number of disease states in which oxidative damage to DNA is suspected (Loft et al., 1992; Loft and Poulsen, 1996, 1998; Helbock et al., 1999).

Isoprostanes and related compounds are of particular interest not only because they are markers for oxidative stress, but because they are biologically active at physiological concentrations (Cracowski et al., 2001; Hou et al., 2004; Montuschi et al., 2004; Roberts et al., 2005). Some isoprostanes are potent vasoconstrictors thereby providing a plausible link between oxidative stress and pathophysiology, for example by raising blood pressure or reducing blood flow, and hence a reduced supply of nutrients to tissues (Cracowski et al., 2001; Hou et al., 2004; Montuschi et al., 2004; Roberts et al., 2005). Indeed, Yao recently proposed that this could provide a mechanism for oxidative stress impacting brain development and function (Yao et al., 2006).

The brain contains the second highest concentration of lipids in the body, after adipose tissue, with 36-60% of nervous tissue being lipids. DHA is the most abundant lipid in the brain (Sastry, 1985). Just as arachidonic acid serves as the precursor for families of enzymatically produced thromboxanes, leukotrienes, prostaglandins and via auto-oxidation, DHA is the precursor of a similar set of molecules including lipoxins and resolvins (Bazan et al., 2005; Serhan, 2005; Bazan, 2006; Serhan et al., 2006) (FIG. 1).

Various diseases, disorders and conditions have been associated with oxidative stress including changes in fatty acids, lipid metabolites and lipid mediators. These diseases include neurological conditions, inflammatory conditions, and cardiovascular or vascular conditions. Among the neurological conditions are autism, Alzheimer's disease, schizophrenia, and Parkinson's disease. Autism (autistic disorder) is a pervasive developmental disorder with diagnostic criteria based on abnormal social interactions, language abnormalities, and stereotypes evident prior to 36 months of age. Despite its lack of Mendelian transmission autism is highly genetically determined.

Children with autistic disorder (AD) show deviation from the normal developmental pattern with impaired social interactions and communication, restricted interests, and repetitive, stereotyped patterns of behaviour that are evident prior to 36 months of age. Clinical genetic studies and modelling studies suggest that AD may be caused by multiple interacting gene loci while environmental and epigenetic factors may contribute to variable expressivity possibly through interaction with genetic susceptibility factors (Muhle, R. et al (2004) 113(5):e472-e486, Szatmari, P. (2003) BMJ 326(7382):173-174, Lawler, C P. et al (2004) Ment Retard Dev Dis Disabil Res Rev 10(4):292-302). Environmental factors contributing to AD could include toxic endogenous metabolites or exogenous toxins or teratogens.

Some recent studies in humans have linked oxidative stress to autism (Chauhan, A. et al (2006) 13(3):171-181). For example, significantly decreased levels of glutathione (GSH), significantly lower ratio of reduced GSH to oxidized GSH, and other metabolic abnormalities in individuals with autism were interpreted as evidence of oxidative stress (James, S J. et al (2004) 80(6):1611-1617, James, S J. et al (2006) 141(8):947-956) Glutathione is the most important endogenous antioxidant and is the most abundant non-protein thiol (Coles, B F et al (2003) 17(1-4):115-130, Li, Y. et al (2004) 66(3):233-242). Recently, increased urinary excretion of 8-isoprostane-F2α, a biomarker of lipid peroxidation and oxidative stress, was found in autism, a finding that has been confirmed (Ming, X. et al (2005) 73(5):379-384, Yao, Y. et al (2006) 63(8):1161-1164).

There remains a need for methods and assays to diagnose, monitor and determine susceptibility to diseases and conditions associated with unresolved or altered oxidative stress. Improved methods and additional relevant biochemical and genetic markers are therefore needed. Further, assessment of relevant and novel targets for intervention and therapy to prevent, alleviate, and modulate these diseases, and a means to monitor the efficiency and manage the effectiveness of intervention and therapy are needed.

The citation of references herein shall not be construed as an admission that such is prior art to the present invention.

### SUMMARY OF THE INVENTION

This invention relates to a system utilizing biochemical markers and genetic markers to diagnose, predict, and/or monitor intervention of a number of diseases and conditions that have unresolved oxidative stress as an important component. The present invention relates generally to markers and assays for diagnosing, predicting, and monitoring disease, particularly disease-relevant oxidative stress and lipid metabolites and mediators.

The invention relates generally to the combined characterization of biochemical markers which assess oxidative stress and the relative levels of lipid and stress mediators and genetic markers associated with altered or increased oxidative stress to diagnose, predict, and/or monitor intervention of a number of diseases and conditions that have unresolved oxidative stress as an important component. Thus, alterations in the detoxification pathway or increased oxidative stress or DNA damage in an individual can result in an increased risk for or susceptibility to various diseases or conditions, including chronic or acute conditions. The biochemical and genetic markers may be utilized in tests, assays, methods, kits for diagnosing, predicting, modulating, or monitoring such diseases or conditions, including ongoing assessment, monitoring, susceptibility assessment, carrier testing and prenatal diagnosis. Management of oxidative stress may be monitored and effects of candidate therapies and therapeutics may be determined by analyzing biochemical markers and determining gene expression.

The present invention therefore provides methods for compiling genetic and biochemical datasets of an individual or individuals for use in determining a disease or condition, assessing its severity, predicting probability or susceptibility for having or developing a disease or condition, or for having offspring that develop a disease or condition.

The invention provides a method for diagnosis or monitoring a disease or condition in an individual comprising:

(a) collecting one or more biological sample from said individual, wherein the biological sample(s) contain proteins, lipids and nucleic acids of the individual;
(b) analyzing the proteins and/or lipids from a biological sample to determine selective metabolites and oxidation products of arachidonic acid (AHA) docosahexanoic acid (DHA) and eicosapentaenoic acid (EPA); wherein said analyzing results in a metabolic determination of oxidative stress and lipids; and
(c) analyzing the nucleic acids from a biological sample to determine the genotype and/or expression of genes involved in oxidative stress and/or lipid metabolism;

wherein the existence or severity of a disease or condition is determined.

The method may further comprise analyzing the nucleic acids from a biological sample to determine the genotype and/or expression of genes associated with or relevant to a selected disease.

In one aspect of the invention, the method involves analyzing the nucleic acid utilizes PCR analysis.

In a further aspect, the method involves analyzing the proteins or lipids utilizes mass spectrometry.

Methods are provided wherein step (b) comprises determining levels of one or more of Resolvins D1-D6, E1 or E2 utilizing chemically synthesized and labeled compounds.

The invention includes methods wherein additional genes associated with a disease selected from autism, Alzheimer's disease, stroke, asthma, multiple sclerosis (MS), inflammatory bowel disease (IBD), cystic fibrosis, rheumatoid arthritis (RA), Parkinson's disease, schizophrenia, brain trauma, BPD, dyslexia, depression, ADHD, cardiovascular disease, atherosclerosis and vascular disease. The invention includes methods wherein additional genes associated with a disease selected from autism, asthma, and Alzheimer's disease are analyzed.

In one such aspect, the disease is autism and the genotype and/or expression of one or more genes set out in Table 4 are determined.

In a further such aspect, the disease is asthma and the genotype and/or expression of one or more genes set out in Table 5 are determined.

In a still further aspect, the disease is Alzheimer's disease and the genotype and/or expression of one or more genes set out in Table 6 are determined.

The invention provides an assay system for diagnosis or monitoring a disease or condition having unresolved oxidative stress as a component which comprises:

(a) collecting a blood, urine or breath sample for biochemical analysis and isolating nucleic acid from said subject;
(b) analyzing the blood, urine or breath sample to determine selective metabolites and oxidation products of arachidonic acid (AHA), docosahexanoic acid (DHA) and eicosapentaenoic acid (EPA); wherein said analyzing results in a metabolic determination of oxidative stress and lipids; and
(c) analyzing the nucleic acids to determine the genotype and/or expression of genes involved in oxidative stress and/or lipid metabolism;
wherein the existence or severity of a disease or condition is determined.

The invention provides a method for monitoring therapeutic intervention of a disease or condition having unresolved oxidative stress as a component which comprises:

(a) collecting a blood, urine or breath sample for biochemical analysis and isolating nucleic acid from said subject;

(b) analyzing the blood, urine or breath sample to determine selective metabolites and oxidation products of arachidonic acid (AHA), docosahexanoic acid (DHA) and eicosapentaenoic acid (EPA); wherein said analyzing results in a metabolic determination of oxidative stress and lipids; and

(c) analyzing the nucleic acids to determine the genotype and/or expression of genes involved in oxidative stress and/or lipid metabolism;

wherein the existence or severity of a disease or condition is determined.

Other objects and advantages will become apparent to those skilled in the art from a review of the following description which proceeds with reference to the following illustrative drawings.

### BRIEF DESCRIPTION OF THE DRAWINGS

FIG. 1 shows DNA-derived metabolites, including inflammatory lipids and anti-inflammatory lipids.

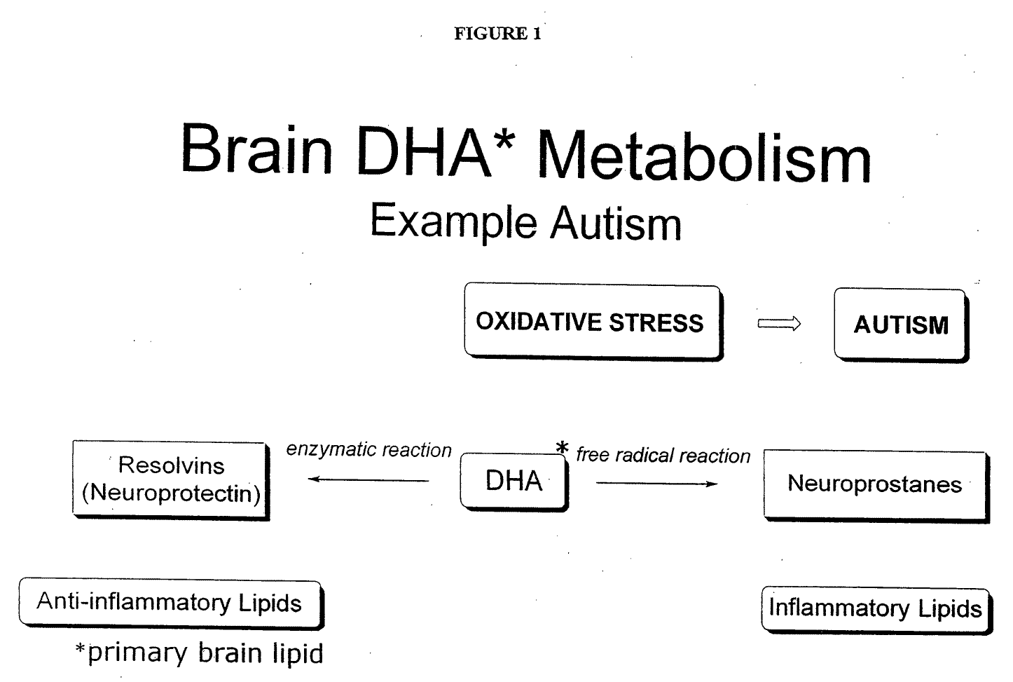

FIG. 2 diagrams the species, substrates, metabolites and markers of oxidative stress and lipid metabolism.

FIG. 3 shows that isoprostane, but not 8-OHdG, is increased in women who will develop preeclampsia.

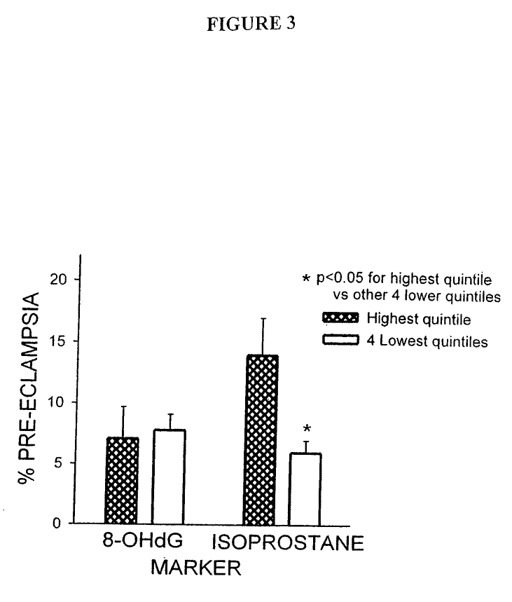

FIG. 4 depicts assessment of isoprostane and 8-OHdG in urine samples of autistic children and healthy controls. Isoprostane is significantly increased in autistic children.

FIG. 5 diagrams membrane bound fatty acids and lipid metabolites and genes involved in the metabolism and conversion to relevant biomarkers.

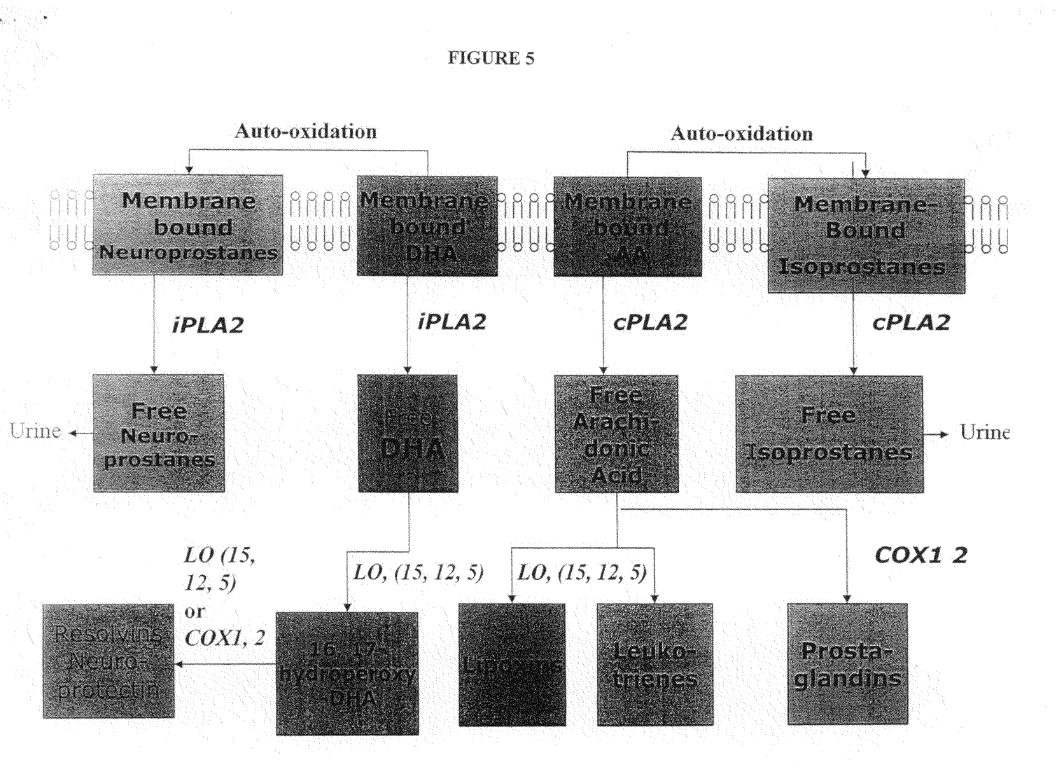

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the terms shall have the definitions set out below.

Various gene names and nomenclature are provided and referred to herein, including in any of the Tables 1-6, and refer to art-recognized nomenclature for these genes and/or their encoded polypeptides. The nucleic acid sequences of these gene(s) and the amino acid sequences of the polypeptide(s) are recognized, known and publically available, including in The National Center for Biotechnology Information (NCBI) and its Genbank database (see e.g. ncbi.nlm.nih.gov). These sequences include sequences of the relevant human gene(s) and other mammalian genes or other orthologs, any of which are available or can readily be determined by the skilled artisan. Similarly, known or identified alleles, including disease relevant or disease-associated alleles, are contemplated herein and included in the gene references as applicable to disease or altered oxidative stress. Alleles contemplate and include any such variants, mutations, alterations, deletions, single nucleotide polymorphisms, RFLPs etc thereof.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

An “upstream regulatory region” is a DNA regulatory region capable of binding RNA polymerase in a cell and initiating transcription of a downstream (3′ direction) coding sequence. For purposes of defining the present invention, the upstream regulatory region sequence is bounded at its 3′ terminus by the transcription initiation site and extends upstream (5′ direction) to include the minimum number of bases or elements necessary to initiate transcription at levels detectable above background and under appropriate regulatory control. Within the upstream regulatory region sequence will be found a transcription initiation site (conveniently defined by mapping with nuclease S1), as well as protein binding domains (consensus sequences) responsible for the binding of RNA polymerase and regulatory regions (consensus sequences) responsible for appropriate regulatory control, including cellular expression, induction of expression, etc. Eukaryotic promoters will often, but not always, contain “TATA” boxes and “CAT” or “CATA” boxes.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 10 or more nucleotides, preferably 15-25 nucleotides, although it may contain fewer nucleotides or more nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

As used herein, “pg” means picogram, “ng” means nanogram, “ug” or “μg” mean microgram, “mg” means milligram, “ul” or “μl” mean microliter, “ml” means milliliter, “l” means liter.

A labeled oligonucleotide or primer may be utilized in the methods, assays and kits of the present invention. The labeled oligonucleotide may be utilized as a primer in PCR or other method of amplification and may be utilized in analysis, as a reactor or binding partner of the resulting amplified product. In certain methods, where sufficient concentration or sequestration of the nucleic acid to be analysed or assessed has occurred, and wherein the oligonucleotide label and methods utilized are appropriately and sufficiently sensitive, the nucleic acid may be directly analyzed, with the presence of, or presence of a particular label indicative of the result and diagnostic of the relevant locus' genotype. After the labeled oligonucleotide or primer has had an opportunity to react with sites within the sample, the resulting product may be examined by known techniques, which may vary with the nature of the label attached. The label utilized may be radioactive or non-radioactive, including fluorescent, colorimetric or enzymatic. In addition, the label may be, for instance, a physical or antigenic tag which is characterized by its activity or binding.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein. Fab and F(ab′)₂ portions of antibody molecules can be prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

This invention relates generally to a system utilizing a combination of biochemical markers and genetic markers to help diagnose, predict, and/or monitor intervention of a number of diseases and conditions that have unresolved oxidative stress as a relevant component. Assays and methods applicable to the system are included in the invention. By combining biochemical and genetic markers, a complete assessment of an individual's response to and ongoing status of oxidative stress can be validly determined.

In the system of the invention, methods are employed to analyze biomarkers, biochemical indicators of oxidative stress, lipid metabolites and lipid mediators. In conjunction with the biomarker assays, genetic markers are determined. Genes involved in or associated with oxidative stress, lipid metabolism, stress response, or stress signaling are analyzed to determine allelic variations or mutations associated with or relevant to oxidative stress, or alterations in stress response, lipid metabolism or relevant signaling. Thus, a combined biochemical and genetic scan of an individual is determined.

Accordingly, it is a principal object of the present invention to provide a method for identifying an individual that is biochemically and genetically inclined to have a disease or condition associated with altered oxidative stress.

It is a further object of the present invention to provide a method for identifying an individual that is genetically inclined to have offspring with a disease or condition associated with altered oxidative stress.

The biochemical markers comprise and involve the simultaneous detection of markers of oxidative stress from arachidonic acid (AA) docosahexaneoic acid (DHA) and eicosapentaenoic acid (EPA). These markers include the Isoprostanes, the urinary Isoprostanes, metabolites and the anti-inflammatory lipid mediator Lipoxins and Resolvins. The measurement of these biochemical markers allows the quantification of diseases and disease stages including the capacity of the human to recover from oxidative stress and lipid metabolites and mediators, including downstream effects on cells and tissues, including but not limited to neutrophils, neurons, glial cells, immune cells. Also, oxidative stress molecules act as signaling molecules themselves and can result in induced damage. The measurement of un-metabolized isoprostane is an indicator of diminished organ specific p-450 metabolism. In the case of neurological diseases, the measurement of the urinary metabolite neuroprostanes and quantification of the anti-inflammatory neuroprotective resolvins and neuroprotectins is relevant to monitoring and/or predicting disease. Further, effects of mediation of oxidative stress and stress effects can be monitored and evaluated upon administration of anti-inflammatory molecules and mediators, including resolvins, lipoxins, and other factors, agents, and compounds.

The genetic markers consist of common variations of small effect many times acting in pathways. Disorders have some pathways in common and some that are specific to that disorder. Combinations of these variations interacting with environmental factors may alter the control (ex: induction, regulation) or metabolism (ex: ability to synthesize or degrade) of the biochemical markers. There are multiple signaling cascades and multiple metabolic pathways relevant to the control/metabolism of these biochemical markers.

Together the biochemical markers and genetic markers will allow the determination of the status of these diseases and the capacity for self control. It will facilitate diagnosis and follow up on any intervention strategy to improve the nature, extent and stage of the disease. Varied types of diseases can be specifically addressed, including but not limited to Autism, Alzheimer's disease, stroke, asthma, multiple sclerosis (MS), inflammatory bowel disease (IBD), cystic fibrosis, rheumatoid arthritis (RA), Parkinson's disease, schizophrenia, brain [symbol] vascular disease.

In one exemplary disease situation, having the products of oxidative stress formed and acting within the brain is likely to be a much more subtle effect than raising blood pressure or generally restricting nutrient supply in genetically susceptible individuals. Autism is a subtle brain disease. The arachidonic acid hypothesis lacks the specificity of the DHA hypothesis. For assessment of oxidative damage to neural tissues, including brain, the assay of DHA metabolites may be more important than the isoprostanes.

Oxidation stress is also relevant in development. Damage to the fetal genome that occurs early in development, when there are fewer cell lines, is likely to apply to those cell lines and their many descendants. Early pregnancy is the time when the fetus is particularly vulnerable to damage from oxidative stress. Oxidative stress that damages the fetus directly or indirectly may be an “explanation” for the fetal origins of adult diseases.

Reports of assessment and involvement of DHA, AA or EPA themselves, or their metabolites and related fatty acids, in various physiologies and pathologies are numerous. These molecules are involved in modulating cell function, membrane function, and signaling. Associations have been established with cancer risk, inflammatory disorders, neurological disorders, atherosclerosis, immune conditions and response, as well as cognition, behavior and mood (Chapkin R S et al Chem Phys Lipids (2008) Epub March 4; Wassail S R and Stillwell W Chem Phys Lipids (2008) Epub February 23; Kidd P M Altern Med Rev (2007) 12(3):2-7-227; Calder P C Prostaglandins Leukot Essent Fatty Acids (2007) 77(5-6):327-335; Li Q et al Molec Immunol (2008) 45(5):1356-1365; Innis S M Early Hum Develop (2007) 83(12): 761-766; Li Q et al Arch Biochem Biophys (2007) 466(2):250-259; Sagaard R et al Biochemistry (2006) 45(43):13118-13129; Muskiet F A and Kemperman R F J Nutr Biochem (2006) 17(11):717-727; Kodas F et al J Neurochem (2004) 89(3):695-702). An understanding and recognition of the relevance of these to development, diseases, conditions, and as continual markers is developing and emerging. The availability of ready and reliable tests and marker assays will facilitate a continued understand and monitoring of these molecules and metabolites.

### I. Biochemical Markers

It is thus an object of the invention to provide analyses for markers of oxidative stress and selective metabolites of Arachidonic acid, Docosahexaenoic acid, and Eicosapentaenoic acid. A standard battery of tests for assessing anti-oxidant status may be used to assess anti-oxidant defense status. These assays include the total peroxyl radical trapping potential (TRAP) in the plasma and specific anti-oxidants. Anti-oxidant defenses fall into two categories, endogenous and exogenous. Key endogenous defenses are the enzymes superoxide dismutase (SOD), catalase, glutathione peroxidase (GPx) and the peptide anti-oxidant is Glutathione (GSSH). The principal dietary (exogenous) originated defenses are vitamins C, β-carotene and vitamin E. To assess oxidative stress in children state of the art assays for plasma malondialdehyde and the urinary excretion of 8-hydroxy-2-deoxyguanosine (8-OHdG), isoprostane (8-iso-PGF_{2α}), the isoprostane metabolite 2,3 Dinor-5,6 dihydro-PGF₂ₜ the ω-3 metabolite iPF_{4α}-VI are used. Since DHA affects the synthesis of anti-inflammatory resolvins, selected resolvins (D1-D6, E1-E2), neuroprotectin and lipoxin A4 are measured. The general approach is to use GC-MS with isotope dilution.

Many of the assays involve GC-MS stable isotope dilution analyses. The major requirement in using isotope dilution selective ion monitoring-GC-MS for measuring biomolecular compounds of interest is the availability of a suitable labeled molecule to serve as the internal standard. MS methods and biosynthesis methods for generating standards are known and several have recently been reported (Hong, S et al J Am Soc Mass Spectrometry (2007) 18:128-144; Lu Y et al (2007) Rapid Commun Mass Spectrom 21:7-22; Masoodi, M et al Rapid Commun Mass Spectrom (2008) 22:75-83).

A measure of DHA relevant oxidative stress can be determined by measurement in urine of F₂-Isop-M, iPF₂ₐ-VI and iPF₄ₐ-VI. Kits for determination of these, as well as 8-isoprostane are available commercially, including from Cayman Chemical, Ann Arbor, Mich.

In addition, neuroprostanes, resolvins and lipoxins can be measured in urine or blood. Methods for detection of these compounds in either urine or plasma are known and/or described (Romano et al., 2002; Gangemi et al., 2003; Chiang et al., 2004; Musiek et al., 2004; Arita et al., 2005; Kadiiska et al., 2005a; Kadiiska et al., 2005b; Morrow, 2005; Lawson et al., 2006; Lu et al., 2006). The general approach includes and involves synthesis of the labeled (²H) and unlabeled compounds and then development of an assay, such as an isotope dilution-GC-MS-selective ion monitoring assay.

Where possible the objective is to provide data for a specific clinical objective from two or more independent assays. The reasons for wanting to doing so are: (i) all of the proposed assays are ‘whole body’ assays based on the measurement on a single tissue (blood) or tissue product (urine) from which extrapolation is made to the neural tissue/brain. With whole body assays it is always a problem whether the level of the parameter measured reflects a local tissue specific effect or is indeed indicative of the body as a whole (Bier, 1989).

Finding similar results with two independent methods supports the argument that what is being measured is a whole body rather than an artifact. (ii) Furthermore replication with two parallel measures of oxidative stress provides security in the analytical methods used, allows for the identification of analytical errors and outliers thereby increasing the confidence in the overall set of biochemical data. In the case of the anti-oxidant defenses the additional data may indicate which anti-oxidants are decreased helping to define a mechanism for future prevention or treatment.

### Preliminary Results:

Levels of isoprostanes have been measured and altered amounts been associated with various diseases. Lipid peroxidation is postulated as contributing to specific aspects of schizophrenia, for instance, and to complications of its treatment. Isoprostanes, particularly 8-isoPGF(2alpha), as measured by immunoassay in urine, was found to be a valuable indicator of oxidative stress in vivo in schizophrenia. Both isoprostanes and thiobarbuturic acid reactive substances (TBARS) were statistically increased in the urine of schizophrenia patients versus control group (Dietrich-Muszalska A, Olas B World J Biol Psychiat (2007) 11:1-7). F2A isoprostane levels have been found to be increased in Alzheimer's patients (Irizarry M C et al Neurodegener Dis (2007) 4(6):403-405).

The prognostic importance of indices for oxidative stress, specifically maternal endogenous anti-oxidant defenses (SOD, GPx, Vitamins C and E) and iron-associated compounds, together with two markers for oxidative stress—the urinary excretion of iPF_{2α}-III (8-iso-PGF_{2α}) and 8-OHdG excretion—on low birth weight and other poor pregnancy outcomes has been evaluated. The samples used for this study were obtained as part of a prospectively study on the effects of maternal nutrition and growth in 1359 generally healthy pregnant women from Camden, N.J. This study was to test the hypothesis that the risk of low birth weight and other poor pregnancy outcomes in low-income and minority women is associated with an increased level of maternal oxidative stress. Camden is one of the poorest cities in the US. Measurements sets were made at entry to care (13.5±3.1 weeks) and at 28 weeks gestation. Infant low birth weight and the frequency of other poor pregnancy outcomes serve as the outcome measures.

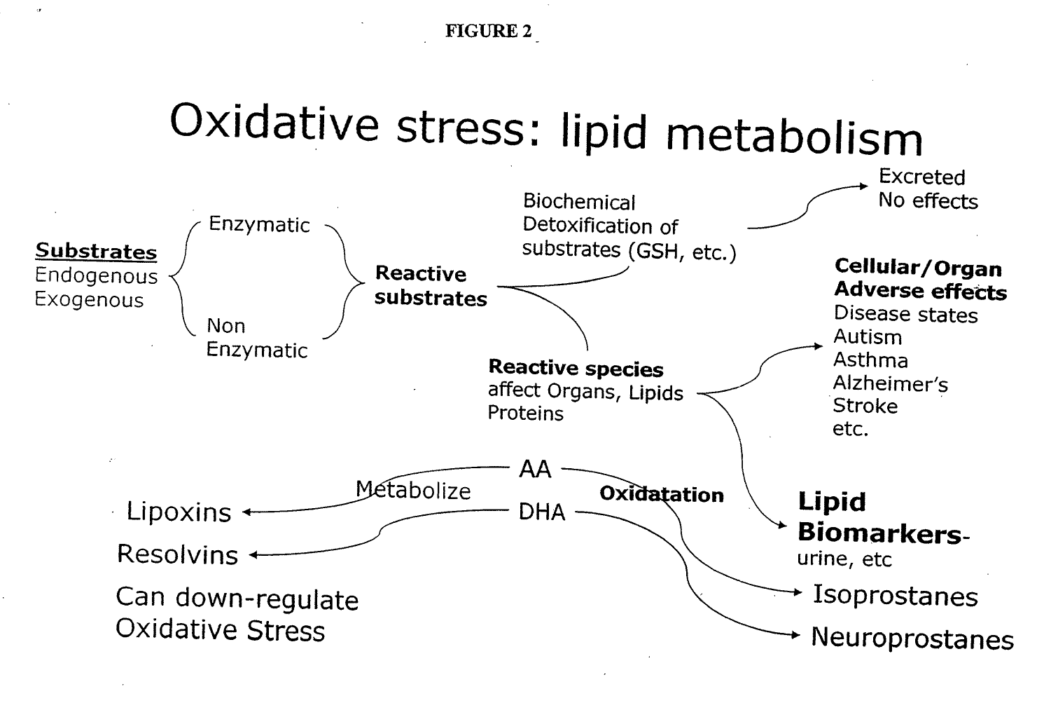

8-OHdG was analyzed by isotope dilution gas chromatography-mass spectrometry (GC-MS) with selective ion monitoring (SIM, (Schwedhelm and Boger, 2003; Il'yasova et al., 2004; Lin et al., 2004). ¹⁸O labeled 8-OHdG was used as the internal standard. Similarly F_{2□} isoprostanes were measured at entry to care GC-MS with isotope dilution and selective ion monitoring. ²H iPF_{2α}-III (²H 8-iso-PGF_{2α}) was used as the internal standard (Stein et al., 2006). To do so we modified a method developed by Lee et al. (Lee et al., 2004; Stein et al., 2006). There was no correlation between the 8-OHdG and iPF_{2α}-III (8-iso-PGF_{2α}) measurements although both were increased with cigarette smoking (Stein et al., 2006). The two markers for oxidative stress (8-OHdG and iPF_{2α}-III_{α}) consistently tracked different maternal anti-oxidant defenses and adverse pregnancy outcomes with the difference being particularly striking for pre-eclampsia (FIG. 2, (Scholl et al., 2005; Stein et al., 2006).

It was concluded that there were two pathways for oxidative stress to affect fetal development. Pathway one, the direct pathway is where there is actual oxidative damage to the fetal DNA. As result the genome is damaged and gene expression impacted. Maternal 8-OHdG excretion, a marker for oxidative damage to DNA, tracks the direct pathway. Pathway two, the indirect pathway, is a consequence of oxidative damage to the mother. As a result the production of F_{2□} isoprostanes is increased. Because these products are biologically active as vasoconstrictors, they impact the supply of nutrients to the fetus. Maternal isoprostane excretion may be monitored to assess the indirect pathway.

In further preliminary results, urinary excretion of 8-isoprostane (8-iso-PGF2α), a lipid peroxidation biomarker, and of 8-hydroxy-2-deoxyguanosine (8-OHdG), a biomarker of DNA hydroxylation and indicator of oxidative damage to DNA, was determined and monitored in autistic children versus healthy controls. Commercial ELISA kits are available for 8-iso-PGF_{2α} (Oxford Biochemicals, Midland, Mich.) and 8-OHdG (Genox Corporation, Baltimore, Md.). Ming et al demonstrated increased excretion of 8-isoprostane in autism patients (Ming, X, et al (2005) Prostaglandins, Leukotrienes and Essential Fatty Acids 73:379-384). Isoprostane is significantly increased in autistic children.

### Assays:

The paragraphs that follow describe exemplary assays and methods which may be used, starting with the individual's anti-oxidant status and concluding with the proposed procedures for assessing oxidative stress.

### Anti-Oxidant Status

To investigate whether diminished host anti-oxidant defenses are a factor in the imbalance between pro- and anti-oxidants, a standard and art-recognized battery of tests for assessing anti-oxidant defenses may be used. Assays for anti-oxidant status are largely based on blood measurements. They include the total peroxyl radical trapping potential (TRAP) in the plasma and specific anti-oxidants. Host anti-oxidant defenses fall into two categories, endogenous and exogenous. Four important endogenous defenses are the enzymes superoxide dismutase (SOD), catalase and glutathione peroxidase (GPx). An important peptide anti-oxidant is Glutathione (GSSH). The principal dietary originated defenses are vitamins C, β-carotene and vitamin E.

### (i) Endogenous Anti-Oxidants

The major endogenous anti-oxidant defenses in the blood are β-carotene, vitamins C and E, protein thiols, glutathione, and bilirubin in plasma and superoxide dismutase, glutathione peroxidase and catalase in the red blood cells. Assays can measure either (i) total anti-oxidant capacity, (ii) groups of anti-oxidants or (iii) individual anti-oxidants. These are all standard assays and are available as commercial kits.

Total anti-oxidant status: The measurement of total anti-oxidant status provides information on an individual's overall anti-oxidant status and this may include anti-oxidants not yet recognized or easily measured (Miller et al., 1993; Shaarawy et al., 1998). We will use the Randox kit, which measures the total amount of chain carrying peroxyl species (Randox Inc., San Francisco Calif., (Miller et al., 1993).

SOD, GPx, Catalase, GSSH, and GSH: For Superoxide Dismutase, Glutathione Peroxidase and Catalase kits sold by Cayman Chemical (Ann Arbor, Mich.) can be used. For Glutathione (total and reduced the kit marketed by Oxford Biochemicals can be used (Oxford, Midland, Mich.).

### (ii) Anti-Oxidants of Dietary Origin

The principle dietary anti-oxidants are vitamins A, C and E.

Ascorbic Acid Plasma vitamin C concentrations can be determined by HPLC using the method of Behrens and Madere (Behrens and Madere, 1979). 100 μl of the sample is neutralized to pH 7.0 and the Dihydroascorbic acid oxidized to ascorbic acid with DL-homocysyteine. 20 μl of a 1:5 dilution of this solution is then chromatographed on a C₁₈ reverse phase column and identified using electrochemical detection.

Vitamins A and E: Vitamins A (Retinoids and Carotenoids) and E (α-tocopherol) can be measured by the HPLC method of Bieri (Bieri et al., 1979). This method gives both vitamins from a single HPLC run. Plasma (100 μl) can be extracted with heptane and applied to a Waters HPLC using a Bondapak C18 (3.9 mm×300 mm) column (Waters Chromatography Corp, Milford Mass.). The samples can be eluted isocratically with a mobile phase of methanol: water (97%:3%) with detection at 313 nm (retinol) and 280 nm (α-tocopherol). For internal standards retinol acetate and α-tocopherol acetate can be used.

(iii) Oxidative Stress

Free radical damage can occur with any biomolecule. Most interest has focused on free radical catalyzed lipid peroxidation and damage to the genome (DNA) (Helbock et al., 1999; Loft and Poulsen, 1999; Morrow et al., 1999). Three sets of assays may be utilized, including 8-OHdG for assessing oxidative damage to DNA, and two sets of assays for assessing lipid peroxidation, malondialdehyde and auto-oxidation of polyunsaturated fatty acids (PUFAs).

### Malondialdehyde (MDA):

The original TBARS method for MDA measures the levels of MDA and other alkenals. MDA has provided much useful information in the past using the so called TBARS assay. The assay has been criticized as being somewhat non-specific. The TBARS method has been widely used but has also been widely criticized as being unspecific (Block et al., 2002). There is now a new method of analyzing for MDA using third derivative spectroscopy which is more specific than the earlier thiobarbituric (TBARS) acid method (Block et al., 2002; Kadiiska et al., 2005a). Third derivative spectroscopy is for MDA is preferable to the older malondialdehyde measurements because the assay is less subject to interference from other aldehydes. A method using individually purchased reagents or a kit marketed by Oxis International (Portland Oreg.) can be used.

### 8-HYDROXY-2-DEOXYGUANOSINE (8-OHdG):

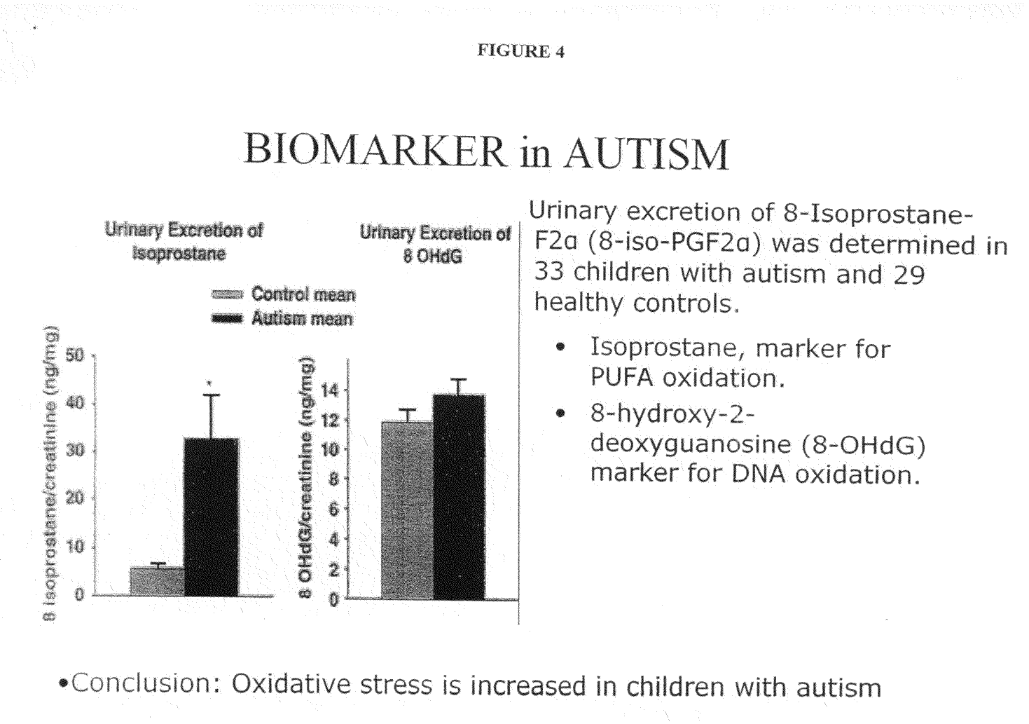

DNA is vulnerable to oxidative damage and therefore extensive repair mechanisms are present in the cell to minimize damage to DNA and repair any damage that does occur. However the repair processes are not 100% efficient and therefore damaged nucleosides accumulate with age in both nuclear and mitochondrial DNA. The products from the oxidative damage of the four DNA bases are not reincorporated into DNA during DNA repair processes, rather they are excreted into the urine without further metabolism (Shigenaga et al., 1989; Loft and Poulsen, 1998). The most abundant of these oxidized nucleosides, 8-hydroxydeoyguanosine is excreted quantitatively in the urine and as such it has been shown to be a marker for DNA damage (Shigenaga et al., 1989; Loft et al., 1995). Increases in 8-hydroxydeoyguanosine excretion correlate with a number of disease states in which oxidative damage to DNA is suspected (Loft et al., 1992; Loft and Poulsen, 1996, 1998; Helbock et al., 1999). In pilot studies non-significant trends towards increased 8-OHdG excretion with autism were found (FIG. 4 and Ming, X et al (2005) Prostaglandins, Leukotrienes and Essential Fatty Acids 73:379-384).

8-OHdG is not only the most abundant oxidative product of cellular DNA oxidation (Ames, 1989; Floyd, 1990) but is also a very a potent mutagen (Ames, 1989; Floyd, 1990; Shibutani et al., 1991; Takeuchi et al., 1994; Lodovici et al., 2000). The concentration of 8-OHdG is increased in tumor-related genes (Kamiya et al., 1992; Takeuchi et al., 1994; Lodovici et al., 2000) and in the DNA of patients with cancer (Kondo et al., 2000; Lodovici et al., 2000; Schwarz et al., 2001; Akcay et al., 2003). Increased DNA bound 8-OHdG has been implicated in a number of other disorders, including neurodegenerative disease (Ferrante et al., 1997; Mecocci et al., 2002), diabetes (Dandona et al., 1996; Leinonen et al., 1997), decreased fecundity (Loft et al., 2003) and pregnancy outcome (Scholl and Stein, 2001). All of these outcomes occur long after the initial oxidative insult. Environmental factors (e.g. pollutants (Marczynski et al., 2000; Toraason et al., 2001; Wako et al., 2001; Zhang et al., 2003) as well as radiation exposure (Povey et al., 1993; Clayson et al., 1994; Plummer et al., 1994; Sperati et al., 1999; Mei et al., 2003) can result in increased 8-OHdG accumulation in DNA. The concentration of 8-OHdG in blood leukocytes increases in proportion to dose (Ames, 1989; Povey et al., 1993; Wilson et al., 1993; Pouget et al., 1999; Cadet et al., 2004).

8-OH G is mutagenic: 8-OHdG is the product of free radical attack on DNA bound guanosine. 8-OHdG is the most abundant oxidative product of cellular DNA oxidation (Ames, 1989; Floyd, 1990). 8-OHdG is a potent mutagen (Ames, 1989; Floyd, 1990; Shibutani et al., 1991; Takeuchi et al., 1994; Lodovici et al., 2000). The accumulation of 8-OHdG in DNA is believed to increase the risk of DNA mutations and cancer development (Akizawa et al., 1994). The concentration of 8-OHdG is increased in tumor-related genes (Kamiya et al., 1992; Takeuchi et al., 1994; Lodovici et al., 2000) and in the DNA of patients with cancer (Kondo et al., 2000; Lodovici et al., 2000; Schwarz et al., 2001; Akcay et al., 2003). Increased DNA bound 8-OHdG has been implicated in a number of other disorders, including neurodegenerative disease (Ferrante et al., 1997; Mecocci et al., 2002), diabetes (Dandona et al., 1996; Leinonen et al., 1997), decreased fecundity (Loft et al., 2003) and pregnancy outcome (Scholl and Stein, 2001). All of these outcomes occur long after the initial oxidative insult. Environmental factors (e.g. pollutants (Marczynski et al., 2000; Toraason et al., 2001; Wako et al., 2001; Zhang et al., 2003) as well as radiation exposure (Povey et al., 1993; Clayson et al., 1994; Plummer et al., 1994; Sperati et al., 1999; Mei et al., 2003) can result in increased 8-OHdG accumulation in DNA. On the ground, radiation increases the concentration of 8-OHdG in blood leukocytes in proportion to dose (Ames, 1989; Povey et al., 1993; Wilson et al., 1993; Pouget et al., 1999; Cadet et al., 2004). Finally there are the various enzymes involved in repairing damaged DNA (Wood et al., 2001) and decreased repair capacity can also result in increased 8-OHdG accumulation (Aburatani et al., 1997; Lu et al., 1997; Radicella et al., 1997).

### Isoprostanes, Neuroprostanes and Resolvins:

Recently a consensus has developed that the measurement of 8-hydroxy-2-deoxyguanosine (8-OHdG) and isoprostanes are preferred markers for oxidative stress (Block et al., 2002; Kadiiska et al., 2005a; Kadiiska et al., 2005b). The F₂ Isoprostanes are derived from the auto-oxidation of arachidonic acid containing phospholipids resulting in a series of PGF₂ like compounds. Excess reactive oxygen species overcome the anti-oxidant defenses and attack polyunsaturated fatty acids such as arachidonate. The resultant bicyclo-endoperoxide prostaglandin intermediates are reduced to four regioisomers each of which can comprise 8 racemic diastereoisomers. These 64 isomers are collectively called the PGF_{2α} isoprostanes. The formation of isoprostanes is independent of the cyclooxygenase enzymes (Roberts et al., 2005). The most studied isoprostane is 8-iso-PGF_{2α} which is also known as iPF_{2α}-III.

As described above, an investigation of the urinary excretion of the isoprostane iPF_{2α}-III (8-iso-PGF_{2α}) and 8-hydroxy-2-deoxyguanosine (8OHdG), in children with Autism and age-matched controls (Ming et al., 2005) has been reported. Others have subsequently confirmed the findings (Yao et al., 2006). A statistically significant increase in isoprostane excretion with Autism was found (Ming et al., 2005).

Experience using kits for 8-OHdG and isoprostane for the pregnancy study showed the kits to unreliable and unsuitable for longitudinal studies. There were large intra-kit differences (up to 20%) and sometimes very large (>100%) differences between batches of kits. This was particularly true for early batches. Hence a switch to isotope dilution—GC-MS assays was made. Correlations between kits and GC-MS were in the 0.5 to 0.6 range.

At the time the pregnancy studies were started (2000), mass spec methods were not recommended because the consensus was that they were very prone to artifacts. These problems were solved in 2003/2004 with the introduction of stable isotope dilution methods for both 8-OHdG and isoprostanes (Schwedhelm and Boger, 2003; Hu et al., 2004; Il'yasova et al., 2004; Lee et al., 2004; Lin et al., 2004; Peoples and Karnes, 2005; Poulsen, 2005; Davies et al., 2006).

Isoprostanes and related compounds are of particular interest not only because they are markers for oxidative stress, but because they are biologically active at physiological concentrations (Cracowski et al., 2001; Hou et al., 2004; Montuschi et al., 2004; Roberts et al., 2005). Some isoprostanes are potent vasoconstrictors thereby providing a plausible link between oxidative stress and pathophysiology, for example by raising blood pressure or reducing blood flow, and hence a reduced supply of nutrients to tissues (Cracowski et al., 2001; Hou et al., 2004; Montuschi et al., 2004; Roberts et al., 2005). Indeed, Yao recently proposed that this could provide a mechanism for oxidative stress impacting brain development and function (Yao et al., 2006).

The metabolic aberrations associated with certain diseases, such as Autism, may likely be small; autistic children are not physically in poor health, the lesion(s) do not threaten physical well being, but rather aspects of behavior and so could be accounted for by minor perturbations in key brain signaling pathways. Therefore, for completeness, a comprehensive series of assays to investigate urinary markers for PUFA oxidation may be implemented. Various assays have been reported or are in development. An assay for iPF_{2α}-III (8-iso-PGF_{2α}) is has been implemented and reported (Stein et al. 2006). Published isotope dilution assays are available for both F₂-Isop-M and iPF_{4α}-VI (Musiek et al., 2004; Lawson et al., 2006); however they are LC-MS-MS based.

Reports on reliable detection of neuroprostanes in human urine are somewhat inconsistent. Mixed neuroprostanes are detectable (Musiek et al., 2004). A different result was reported by Lawson et al. (Yao et al., 2006). They elected to focus on group VI F₄-neuroprostanes because among AA derived isoprostanes, group VI isoprostanes are the most abundant in human urine (Yao et al., 2006). They argued that because of the close structural analogies between iPF_{3□}-VI 3 and nPPF_{4□}-VI, it was likely that this could also be the case for group VI F₃-iPs and F₄-nPs. However even with their state of the art LC-MS-MS they were unable to detect any of the expected neuroprostane nPF_{4□}-VI (Lawson et al., 2006). Further investigations in rats showed why. The DHA derived neuroprostanes were rapidly oxidized by the liver (Lawson et al., 2006). In contrast the AA derived analog was not. They attributed the difference in behavior between the isoprostanes and neuroprostanes to the presence of a hydroxyl group in the 5 position of the isoprostanes which confers resistance to oxidation (Pratico et al., 2004; Lawson et al., 2006). As a result neuroprostanes such as nPF_{4□}-VI are oxidized in the liver to the stable end product iPF_{4□}-VI. iPF_{4□}-VI is very abundant in the urine, (200-400 ng mg Creatinine⁻¹, (Yao et al., 2006).

However iPF_{4□}-VI can also be formed direct oxidation of EPA as well as by □-oxidation of the DHA derived neuroprostane nPF_{4□} (Lawson et al., 2006). Yao et al concluded that (iPF_{4□}-VI) is ‘an excellent marker for the oxidation □3-PUFAs (EPA+DHA)’ (Yao et al., 2006). Likewise, the urinary metabolite of iPF_{2α}-III, F₂-Isop-M, is an excellent marker for AA oxidation (Roberts et al., 1996). Thus analysis of the urinary excretion of these two metabolites can provide an indication of (i): whether there is a general increase in the auto-oxidation of PUFAs and (ii) whether there is increased production of iPF_{4□}-VI (from EPA+DHA). Since DHA is more abundant than EPA and the major location of DHA is neural tissue, it is not unreasonable to interpret an increase in iPF_{4□}-VI excretion as being due to increased oxidative damage to brain lipids (Sastry, 1985; Yao et al., 2006).

Yao found that not only was production of the AA derived isoprostane iPF_{2α}-VI increased, so were several other AA derived metabolites (leukotrienes, thromboxanes (Yao et al., 2006). The perturbation of AA metabolism appeared to be more widespread than just an increase in the auto-oxidation of AA. Indeed, a very recent report from Austria of a pilot study with DHA supplementation suggested that there were positive benefits to DHA supplementation for autism (Amminger et al., 2006). Collectively our data, Yao's data and the recent Austrian study support providing the clinical team within reason, as extensive a series of assays of DHA metabolites as possible (Amminger et al., 2006; Stein et al., 2006; Yao et al., 2006).

DHA, in addition to being the precursor for neuroprostanes, is the precursor of large families of enzymatically derived bioactive anti-inflammatory mediators, the resolvins, docosatrienes and neuroprotectins (Serhan, 2005; Bazan, 2006). These molecules are involved in signal transduction processes and have been shown to have potent anti-inflammatory protective and neuroprotective properties (Serhan, 2005; Bazan, 2006). If result similar to those found by Yao for AA are found with DHA, the generation of small amounts of ‘unusual compounds (neuroprostanes etc.)’ which are chemically and sterically similar to enzymatically derived metabolites of DHA would have the potential to interfere with normal brain signal transduction pathways.

Recently, Serhan et al. reported a new class of lipid mediators derived from docosahexaenoic and eicosapentaenoic acid that posses potent anti-inflammatory and immunoregulatory activities in the low picomolar to nanomolar range ((Schwab J M, Serhan C N Curr Opin Pharmacol (2006) 6 (4): 414-420; Arita M et al Prostaglandins & Other Lipid Med (2006) 79 (1-2): 154-154; Serhan C N et al J Immunol (2006) 176 (3): 1848-1859; Arita M, Clish C B, and Serhan C N B B Res Commun (2005) 338 (1): 149-157; Arita M, et al PNAS USA (2005) 102 (21): 7671-7676; Flower R J, Perretti M J Exp Med (2005) 201 (5): 671-674; Serhan C N et al Lipids (2004) 39 (11): 1125-1132; Serhan C N, et al Prostaglandins & Other Mediators (2004) 73 (3-4): 155-172; Bazan N G Molec Neuro (2005) 31 (1-3): 219-230; Serhan C N et al Prostaglandins & Other Mediators (2004) 73 (3-4): 155-172). These new compounds are formed in vivo via cell-cell interaction and were named Resolvins (resolution phase interaction products). Docosahexaenoic acid is highly enriched in brain, synapses and retina. Deficiencies of this ω-3 fatty acid are associated with Alzheimer's disease, stroke, hyperactivity, schizophrenia and peroxisomal disorders. Other diseases that are associated with diminished formation of these “good lipid mediators” are asthma, kidney diseases, inflammatory bowel disease, rheumatoid arthritis, sepsis and other neutrophil-driven diseases. Serhan's work has established the molecular basis and the mechanism of the immune protective action conferred by ω-3 fatty acids.

### Assay Development Methodology

Urine from healthy adults can be used. An approach reported for isoprostanes is implemented (Lee et al., 2004; Stein et al., 2006), namely to take 1 ml of urine, add 50 ng of the deuterated internal standard run it through a Waters SPE-Oasis cartridge (Waters Inc., Milford, Mass.), wash with NH₄OH (2%, 2 ml), 20 mM formate in methanol (2 ml), 100% hexane (2 ml) and elute with ethyl acetate (2 ml), dry under N₂, esterify with N,N-diisopropylenediamine (DIPEA, 15 μl) and 30 ml pentafluorobenzylbromide (PFBBr, 30 ml) in acetonitrile at room temperature for 30 min. The samples are then dried under N₂ and 20 μl of acetonitrile and 40 μl N.O-bis(trimethylsily-)trifluoroacetamide (BSFFA)+15 μl trimethyl-chlorosilane (TMCS) added and the mixture incubated at 40° C. for 1 h and then injected into the GC-MS (Lee et al., 2004; Stein et al., 2006).

### Organic Syntheses

Compounds for use in assays of lipid metabolites and mediators, including resolvins D1-D6, E2, may be generated by recognized and available biosynthesis methods (see eg Serhan work and references as noted above). Alternatively, total chemical synthesis of these compounds may be undertaken. Advantages of total chemical synthesis include reduced costs and enhanced purity. Since only tiny amounts of the Resolvins and such other lipid mediators are available from natural sources, these lipid mediators would be best prepared by total chemical synthesis in order to expedite continuing biological and pharmacological investigations. Spur and Rodriguez have developed methods for synthesis of various resolvins (Rodriguez A R, Spur B W Tetrahedron Letters (2004) 45 (47): 8717-8720; Rodriguez A R, Spur B W Tetrahedron Letters (2005) 46 (21): 3623-3627). In addition, methods for synthesis of resolvins and key intermediates are provided in U.S. Patent Ser. No. 60/920,112, filed Mar. 26, 2007, and corresponding PCT filed Mar. 26, 2008, which are incorporated herein by reference. Spur and Rodriguez detail therein methods to prepare Resolvin D6 (4,17-dihydroxy-5E,7Z,10Z,13Z,15E,19Z-docosahexaenoic acid), and methods for the preparation of isotopically labeled ω-3 fatty acid metabolites, including d4-7(S), 17(S)-Resolvin D5 (10,11,13,14-tetradeutero-7(S),17(S), 4Z,9E,10Z,13Z,15E,10Z docosahexaenoic acid). Certain exemplary and supplemental chemical synthesis methods are also diagrammed herein below.

Exemplary synthetic routes for the chemical synthesis of compounds which are suitable for assays are outlined below. Making the deuterium analogs can be accomplished, for instance, by starting with deuterium labeled intermediates obtained via Lindlar reduction with deuterium gas or from triple bond analogs of resolvins via Zn/Cu/Ag reduction with ²H₄ methanol and ²H₂O. All compounds synthesized can be checked for purity by ¹H-NMR, ¹³C-NMR, UV, FT-IR and HPLC-MS using standard and recognized methods.

### Synthesis of Resolvin D1.

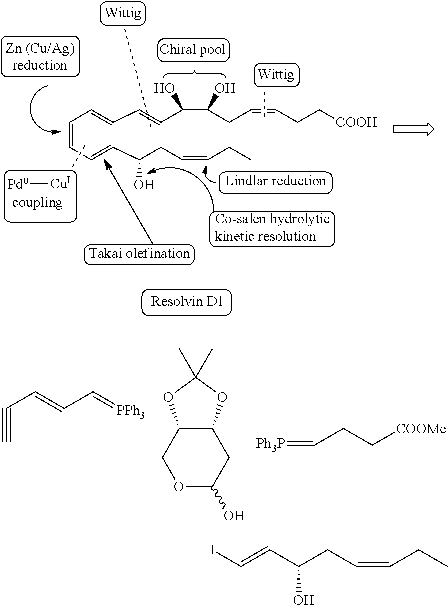

The synthesis of Resolvin D1 will be accomplished similar to our first synthesis of Resolvin D2 (Rodriguez and Spur, 2004, 2005) from 2-deoxy-D-ribose via Wittig reaction, Pd/Cu coupling and Zn/Cu/Ag reduction.

### Synthesis of Resolvin D4

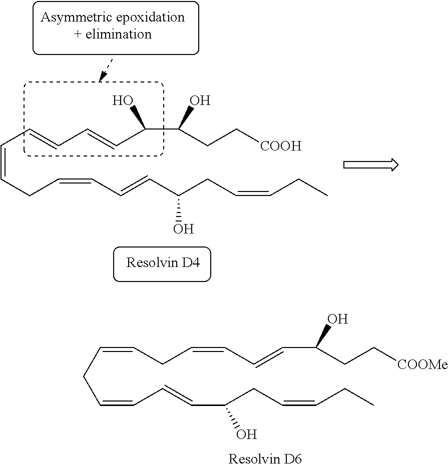

The synthesis of Resolvin D4 will be accomplished from Resolvin D6 via a two-step sequence: a) asymmetric epoxidation and b) based catalyzed epoxide opening to generate Resolvin D4.

### Synthesis of Resolvin D6

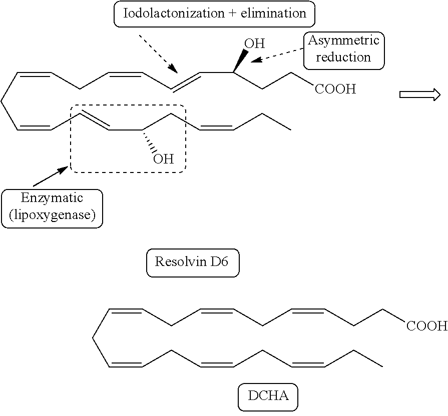

The synthesis of Resolvin D6 will be accomplished from docosahexaenoic acid via enzymatic lipoxygenation, to introduce the 17(S)-hydroxy group, followed by direct iodolactonization and HI-elimination to produced the epimeric Resolvin D6 [4(S and R)—OH]. Chiral-HPLC can separate the two epimers to give Resolvin D6. An alternative route using a mild oxidation of the 4-hydroxy group followed by a stereoselective reduction will provide Resolvin D6 without the need of chiral separation.

The synthesis of Resolvin E2 will be accomplished similar to our first

### Resolvin E2

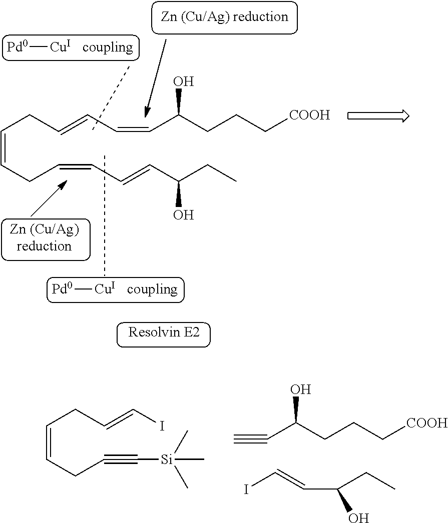

synthesis of Resolvin D5 (Rodriguez and Spur 2005) via two Pd/Cu coupling and the Zn/Cu/Ag reduction of the two triple bonds. The Co-salen hydrolytic kinetic resolution will be used to generate the chiral centers with >99% ee. (Rodriguez and Spur 2003)
E-8. d4-Resolvin E2

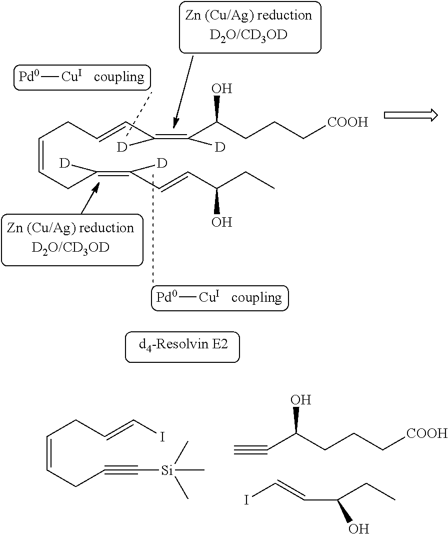

The synthesis of d₄-Resolvin E2 will be accomplished from the same triple bond intermediate used to produce Resolvin E2 but employing Zn/Cu/Ag reduction in D₂O/d₄-MeOH. Lindlar reduction of the triple bonds intermediates using deuterium gas can be used as an alternative.

### Choice of Urine as Assay Fluid

For both 8-OHdG and isoprostanoids there are technical advantages to performing the assays on urine samples. When measured in plasma, some of these products are subject to auto-oxidized by the many pro-oxidants present in plasma; isolating them in a pure enough state often introduces artifacts (Loft and Poulsen, 1998; Morrow and Roberts, 1999). Autoxidation is not a problem with urine because, unlike plasma, urine does not contain a wealth of precursors and catalytic agents. In addition, urine is much less complex chemically so the pre-assay purification steps are fewer and easier to do. Furthermore urine assays give a time-integrated value that can be normalized to creatinine (a measure of body composition) so that there is less variation than with a single spot sample of plasma. Similar arguments are likely to apply to the auto-oxidation products of DHA. Finally for studies on children serial measurements on urine are much more acceptable to the subjects than serial blood collections.

Ancillary assay: Creatinine: Creatinine will be measured the picric acid method using a the procedure as previously described (Stein et al., 1996). The creatinine assay is needed to normalize all of the urine data to creatinine excretion.

### Specimen Collection and Storage

Blood: Blood samples can be collected at in a 3 ml Vacutainer tubes containing lithium heparin ate (Becton-Dickinson, N.J.). The tubes are immediately covered in aluminum foil and stored in the dark at 4° C. until the plasma can be separated. (i) After spinning the blood at 1000 g for 15 minutes the serum and plasma can be removed. (ii) For the ascorbic acid assays 0.7 ml of plasma can be pipetted into blue capped Vanguard Cryogenic vials (Sumitomo Bakelite Co., Neptune N.J.) containing 0.7 ml of a 10% solution of metaphosphoric acid (Comstock et al., 1995) and then stored at −70° C. (iii) The remainder of the plasma can be frozen and stored at −70° C. A study by Comstock et al. showed that plasma ascorbic acid, carotenoids, retinoids and tocopherols were stable if prepared and stored in this way for at least four years (Comstock et al., 1995). (iii) The residual erythrocytes from the heparinized blood can be washed three times with 0.9% NaCl and centrifuged after each wash at 800 g for 7 minutes. 1000 μl of washed cells canl be removed for preparation of ghost free hemolysates. To 1000 μl of washed cells can be added 5 ml cold distilled water containing 0.5% (v/v) Triton-X100. After vortexing the mixture can be centrifuged at 10,500 g for 5 minutes. The upper aqueous phase (the hemolysate) can be removed and stored, wrapped in aluminum foil at −70° C. until analyzed for SOD, catalase, GPX, GSH and GSSH.

Urine: 10 ml of urine can collected in metal free plastic containers and stored at −70° C. Since transition metals can generate oxygen radicals leading to an artifactual increase in 8-OHdG levels, urine can be collected in plastic containers. Adding anti-oxidant stabilizers to the urine is contra-indicated because of the potential of altering the oxidative potential in the specimen. Studies have shown that both 8-hydroxydeoyguanosine and 8-iso-PGF_{2α} are stable for at least a year if stored under these conditions (Tagesson et al., 1992; Rokach et al., 1997).

Other Metabolites Relevant to Oxidative Stress and Lipid Metabolism as Biomarkers

Various other metabolites or molecules relevant to and indicative or oxidative stress or a stress-mediated response may be determined using standard and recognized methods in the art. These methods include direct and/or indirect measurement. Therefore, any of, including one or more, or several of the following may be determined: Glutathione, including GSH and/or GSSG; Thioredoxin, oxidized and/or reduced; Glutaredoxin, oxidized and/or reduced; adenosine; methionine; SAH; SAM; homocysteine; cysteine; cystothionine; cysteinyl glycine; cystine; glucuronic acid; PAPS; Tbars; isoprostanes; neuroprostanes; lipoxin; neuroprotectins; prostaglandins; leukotrienes; AA; DHA and EPA. In addition, or in combination, the levels of pro-inflammatory cytokines such as TNF-α, IL-1β, and IL-6 may be measured in monitoring stress. Also, nitric oxide (NO) may be measured indirectly or directly.

### I. Gene Analyses

The following genes are relevant to oxidative stress, lipid mediators, and lipid metabolism and may be generally applicable as markers to various stress-associated or stress-exacerbated or stress-mediated diseases or conditions. These markers provide a general or generic set for analysis in various conditions, states, or scenarios, including in diagnosing, monitoring, predicting disease or evaluating disease mediation(s).

### A. Oxidative Stress, Lipid Metabolism Genes

Genes Relevant to Biochemical Control and Metabolism.

### Phospholipases A2, Lipoxygenases (LOs), Cyclooxygenases (COXs) and Related Genes

Lipoxins are a series of anti-inflammatory mediators. Their appearance in inflammation signals the resolution of inflammation. Lipoxins are derived from arachidonic acid, an omega-6 fatty acid. An analogous class, the resolvins, is derived from DHA and EPA, omega-3 fatty acids. The calcium-independent phospholipases, PLA2G6 and, PLA2G4C are necessary to release DHA from cell membranes. This is the first step in synthesis of resolvins. The lipoxygenases (LO) ALOX5 along with its activating factor ALOX5AP [FLAP], ALOX15, and ALOX12 are necessary for the production of the anti-inflammatory molecules 16,17-epoxyDHA and resolvins as well as the production of the anti-inflammatory molecule lipoxin and the pro-inflammatory leukotrienes. Cyclo-oxygenases PTGS1 (COX1) and PTGS2 (COX2) are key enzymes in the production of prostaglandins and act as an alternate route of production of resolvins from 16,17-hydroperoxy DHA.

TABLE 1
Genes directly related to DHA/AA/EPA metabolism

| | |
|---|---|
| GLUTATHIONE PEROXIDASE | GPX1 |
| CYCLOOXYGENASE 1; COX1 | PTGS1 |
| CYCLOOXYGENASE 2; COX2 | PTGS2 |
| ARACHIDONATE 5-LIPOXYGENASE; | ALOX5 |
| ARACHIDONATE 12-OXIDOREDUCTASE; | ALOX12 |
| ARACHIDONATE 15-LIPOXYGENASE; | ALOX15 |
| ARACHIDONATE 15-LIPOXYGENASE, SECOND | ALOX15B |
| TYPE; | |
| ARACHIDONATE 12-LIPOXYGENASE, R TYPE; | ALOX12B |
| ARACHIDONATE 5-LIPOXYGENASE-ACTIVATING | ALOX5AP |
| PROTEIN; | |
| ARACHIDONATE LIPOXYGENASE 3; | ALOXE3 |
| FORMYL PEPTIDE RECEPTOR-LIKE 1 (Lipoxin | FPRL1 |
| A4 receptor, ALXR) | |
| PHOSPHOLIPASE A2, GROUP IIA; | PLA2G2A |
| PHOSPHOLIPASE A2, GROUP IB; | PLA2G1B |
| PHOSPHOLIPASE A2, GROUP X; | PLA2G10 |
| PHOSPHOLIPASE A2, GROUP IVA; | PLA2G4A |
| PHOSPHOLIPASE A2, GROUP VII; | PLA2G7 |
| PHOSPHOLIPASE A2, GROUP IVB; | PLA2G4B |
| PHOSPHOLIPASE A2, GROUP VI; | PLA2G6 |
| PHOSPHOLIPASE A2, GROUP IVC; | PLA2G4C |
| PHOSPHOLIPASE A2 RECEPTOR 1; | PLA2R1 |
| PHOSPHOLIPASE A2-ACTIVATING PROTEIN; | PLAA |
| PHOSPHOLIPASE A2, GROUP V; | PLA2G5 |
| PHOSPHOLIPASE A2, GROUP IID; | PLA2G2D |
| ANNEXIN A2; | ANXA2 |
| ANNEXIN A1; | ANXA1 |
| MAP KINASE-ACTIVATING DEATH DOMAIN; | MADD |
| LEUKOTRIENE C4 SYNTHASE; | LTC4S |
| PHOSPHOLIPASE C, BETA-2; | PLCB2 |
| PHOSPHOLIPASE C, GAMMA-2; | PLCG2 |
| PHOSPHOLIPASE C, GAMMA-1; | PLCG1 |
| PHOSPHOLIPASE C-LIKE 1; | PLCL1 |
| PHOSPHOLIPASE C, BETA-3; | PLCB3 |
| PHOSPHOLIPASE C, EPSILON-1; | PLCE1 |
| PHOSPHOLIPASE C, DELTA-1; | PLCD1 |
| PHOSPHOLIPASE C, BETA-4; | PLCB4 |
| PHOSPHOLIPASE C, BETA-1; | PLCB1 |
| PHOSPHOLIPASE C, DELTA-4; | PLCD4 |
| p21-ACTIVATED KINASE- AND PHOSPHOLIPASE | PIP1 |
| C-INTERACTING PROTEIN 1 | |
| PHOSPHOLIPASE C, ZETA-1; | PLCZ1 |
| PHOSPHOLIPASE C, DELTA-3; | PLCD3 |
| PEROXISOME PROLIFERATOR-ACTIVATED | PPARA |
| RECEPTOR-ALPHA | |
| S100 CALCIUM-BINDING PROTEIN A10 | S100A10 |

[symbol]

GSH is a key substrate for detoxification of xenobiotics, metabolites and toxins through the GST pathway as well as a key element in pathways protecting against oxidative stress and maintaining the redox state. GSH is reduced in Autism. In addition the GSH:GSSG ratio is lower. A possible contributing factor for low levels of GSH could be decreased GSH synthesis. GSH synthesis occurs through a multienzyme pathway beginning with the amino acid, cysteine. Low GSH increases JNK and p38 activity. GSTP1 binds to and inhibits JNK. Increased oxidative stress disassociates GSTP1 from JNK. Variations in the promoters of GCLM and GCLC reduce their oxidative stress up-regulation.

Glutamate-cysteine ligase, GCL (also known as gamma-glutamylcysteine synthase) is the rate-limiting enzyme of GSH synthetase. Its two subunits, the catalytic subunit, GCLC, and modifying subunit, GCLM, are coded for by different genes. Varianent in the promotors of both GCLC and GCLM may suppress the oxidant-induced response of the GCLC gene. Both subunits of GCL are also upregulated by AP-1 through JNK and ERK activation. GSH depletion has been shown to activate JNK through a feedback mechanism that leads back to GCL activation

GCLC, GCLM, GSR are upregulated by DHA through JNK. Activity of GST's may also be upregulated by DHA.

Glutathione reductase, GSR, increases GSH by reducing oxidized glutathione, GSSG, to GSH and thus increases the GSH/GSSG ratio in concert with GLRX as discussed below. GSR is important for redox homeostasis; overexpression of GSR attenuates induction of JNK.

Cystathione beta-synthase, CBS, converts the sulfur-containing amino acid homocysteine to cystathionine. CBS is important for GSH synthesis because this pathway produces about 50% of the body's cysteine that ends up in GSH.

Gamma-glutamyltransferase-1, GGT1, is a required step for GSH production in neurons because cystathionine-gamma-lyase (CTH) is not expressed in brain cells. Thus cysteine, and consequently GSH, cannot be synthesized in brain cells by the usual pathway that involves CTH. To synthesize GSH, neurons must take up cysteine (the reduced form of cystine) from which they are able to synthesize GSH. However, cystine not cysteine is transported from blood into brain. Neurons cannot take up cystine but astrocytes can. Consequently, astrocytes take up cystine, use it to synthesize GSH and export the GSH. GGT1 in extracellular fluid hydrolyzes GSH to cysteine, which is then taken up by neurons, which use the cysteine to synthesize the required GSH. GGT is upregulated by the MAPKs, ERK and p38.

The rationale for studying genes related to GSH synthesis is that polymorphic alleles of multiple proteins may decrease GSH synthesis and contribute to the decreased GSH levels observed in autism. These decreased GSH levels could contribute to impairment of GST function in concert with polymorphic variations of GST enzymes themselves. Decreased GSH levels could also lead to impaired responses to oxidative stress and altered MAPK activity.

Thioredoxin (TXN or TRX) is a small cytosolic enzyme with oxidoreductase activity that contains a dithiol-disulfide active site. It contributes to maintaining protein stability under conditions of oxidative stress. TXN binds to the N-terminal region of ASK1 in a fashion highly dependent on the redox status of TRX. When TXN is expressed, ASK1 kinase activity and ASK1-dependent apoptosis are inhibited. Thus, when reduced TXN binds to ASK1, ASK1 is inactive but when TXN is oxidized, the complex breaks up and ASK1 returns to activity. TXN2 is a mitochondrial form coded for by a different gene.

Glutaredoxin (GLRX, GRX or thioltransferase) is a small cytosolic enzyme that catalyzes GSSG oxidoreduction reactions in the presence of glutathione reductase (discussed above) and NADPH; it also acts as a GSH-dependent hydrogen donor for ribonucleotide reductase. Like TRX, GLRX may be a sensor molecule that recognizes oxidative stress. Like TXN, GLRX binds to ASK1 but to the C-terminal region instead. GLRX inhibits ASK1 when it is bound but when released allows ASK1 activation. GLRX and TXN are released from ASK1 by different mechanisms as is GSTM1 by still a third mechanism. However, all three participate in ASK1 regulation.

TABLE 2
Glutathione synthesis and redox maintainence.

| | |
|---|---|
| GLUTAMATE-CYSTEINE LIGASE, MODIFIER SUBUNIT | GCLM |
| GLUTAMATE-CYSTEINE LIGASE, CATALYTIC SUBUNIT | GCLC |
| CYSTEINE DIOXYGENASE, TYPE I | CDO1 |
| GLUTATHIONE REDUCTASE | GSR |
| GLUTATHIONE SYNTHETASE | GSS |
| CYSTATHIONINE GAMMA-LYASE | CTH |
| GAMMA-GLUTAMYLTRANSFERASE 1 | GGT1 |
| GAMMA-GLUTAMYLTRANSFERASE 2 | GGT2 |
| ADENOSINE A2 RECEPTOR | ADORA2A |
| S-ADENOSYLHOMOCYSTEINE HYDROLASE (SAHH) | AHCY |

Additional genes that affect detox/redox state.
Antiox/detox.

| | |
|---|---|
| GLUTATHIONE S-TRANSFERASE, ALPHA-1 | GSTA1 |
| GLUTATHIONE S-TRANSFERASE, ALPHA-4 | GSTA4 |
| GLUTATHIONE S-TRANSFERASE, ZETA-1 | GSTZ1 |
| GLUTATHIONE S-TRANSFERASE, theta-1 | GSTT1 |
| GLUTATHIONE S-TRANSFERASE, MU-1 | GSTM1 |
| GLUTATHIONE S-TRANSFERASE, MU-2 | GSTM2 |
| GLUTATHIONE S-TRANSFERASE, MU-3 | GSTM3 |
| GLUTATHIONE S-TRANSFERASE, MU-4 | GSTM4 |
| GLUTATHIONE S-TRANSFERASE, MU-5 | GSTM5 |
| GLUTATHIONE S-TRANSFERASE, Pi-1 | GSTP1 |
| N-ACETYLTRANSFERASE 1 | NAT1 |
| N-ACETYLTRANSFERASE 2 | NAT2 |
| CYTOCHROME P450, SUBFAMILY IID, POLYPEPTIDE 6 | CYP2D6 |
| CYTOCHROME P450, SUBFAMILY I, POLYPEPTIDE 1 | CYP1A1 |
| CYTOCHROME P450, SUBFAMILY I, POLYPEPTIDE 2 | CYP1A2 |
| CYTOCHROME P450, SUBFAMILY IIA, POLYPEPTIDE 13 | CYP2A13 |
| CYTOCHROME P450, SUBFAMILY IIA, POLYPEPTIDE 6 | CYP2A6 |
| CYTOCHROME P450, SUBFAMILY IIIA, POLYPEPTIDE 4 | CYP3A4 |
| CYTOCHROME P450, SUBFAMILY IIE | CYP2E |
| CYTOCHROME P450, SUBFAMILY IIC, POLYPEPTIDE 8 | CYP2C8 |
| CYTOCHROME P450, SUBFAMILY IIC, POLYPEPTIDE 9 | CYP2C9 |
| CYTOCHROME P450, SUBFAMILY IIJ, POLYPEPTIDE 2 | CYP2J2 |
| UDP-GLYCOSYLTRANSFERASE 1 FAMILY, POLYPEPTIDE A1 | UGT1A1 |
| UDP-GLYCOSYLTRANSFERASE 1 FAMILY, POLYPEPTIDE A3 | UGT1A3 |
| UDP-GLYCOSYLTRANSFERASE 1 FAMILY, POLYPEPTIDE A6 | UGT1A6 |
| UDP-GLYCOSYLTRANSFERASE 1 FAMILY, POLYPEPTIDE A7 | UGT1A7 |
| UDP-GLYCOSYLTRANSFERASE 1 FAMILY, POLYPEPTIDE A8 | UGT1A8 |
| UDP-GLYCOSYLTRANSFERASE 1 FAMILY, POLYPEPTIDE A9 | UGT1A9 |
| UDP-GLYCOSYLTRANSFERASE 2 FAMILY, MEMBER B7 | UGT2B7 |
| UDP-GLYCOSYLTRANSFERASE 2 FAMILY, MEMBER B28 | UGT2B28 |
| SULFOTRANSFERASE FAMILY 1A, PHENOL-PREFERRING, MEMBER 1 | SULT1A1 |
| SULFOTRANSFERASE FAMILY 1A, PHENOL-PREFERRING, MEMBER 2 | SULT1A2 |
| SULFOTRANSFERASE FAMILY 4A, MEMBER 1 | SULT4A1 |
| SUPEROXIDE DISMUTASE 1 | SOD1 |
| SUPEROXIDE DISMUTASE 2 | SOD2 |
| SUPEROXIDE DISMUTASE, EXTRACELLULAR | SOD3 |
| CATALASE | CAT |
| THIOREDOXIN | TXN |
| THIOREDOXIN REDUCTASE 1 | TXNRD1 |
| GLUTAREDOXIN | GLRX |
| HEAT-SHOCK 70-KD PROTEIN 1A (HSP72) | HSPA1A |
| PARAOXONASE 1 | PON1 |
| PARAOXONASE 2 | PON2 |
| PARAOXONASE 3 | PON3 |

### B. Stress Signaling Genes

The following exemplary genes and pathways are relevant to stress signaling. They are important for immune function, detoxification and antioxidants/antioxidation. They also have function in neural development, receptor signaling and apoptosis. These genes are relevant for regulation of the lipid genes and genes involved in lipid mediation.

### Activators & Inhibitors of PLA2s

### MAPKs (MAPKs Control PLA2, LO and COX Activity)

JNK1, JNK2 and JNK3 phosphorylate & activate PLA2s, both calcium-dependent and calcium-independent forms. JNK1 is inhibited by GSTP1 and HSP72. GSTP1 is associated with autism. JNK1 is developmentally expressed in brain. H-Ras (previously associated with autism) expression up-regulates COX-2 and 12-LO via JNK and ERK. COX-2 expression is predominantly regulated by ERK and JNK. DHA and EPA diminish p38 and JNK and increase ERK activity in cells treated with TNF-alpha. AA metabolites activate JNK, ERK and p38. H₂O₂ increases AA release and increases PLA2 activity followed by MAPK activation. PLA2 inhibitors decreased the H₂O₂ stimulation of ERK and JNK. A 5-LO inhibitor prevented JNK stimulation. GSH synthesis is upregulated through JNK by both 4-FINE and DHA showed that oxidized LDL increases JNK activation.

JNK2 and JNK3 are developmentally expressed in brain and may also be inhibited by GSTP1 and HSP72. GSTP1 has been associated with autism.

p38 phosphorylates & activates PLA2s, both calcium-dependent and calcium-independent. The prostaglandin synthesis cascade is in part regulated by p38. p38 is upregulated by AA metabolites, and DHA can attenuate TNF-alpha activation of p38. The anti-inflamatory effects aspirin-triggered lipoxin A4-stable analog are exerted at least in part by blocking the p38 cascade.

ERK1 phosphorylates & activates PLA2s, both calcium-dependent and calcium-independent. H-Ras (previously associated with autism) expression up-regulates COX-2 and 12-LO via JNK and ERK. COX-2 expression is predominantly regulated by ERK and JNK. AA metabolites activate INK, ERK and p38. PLA2 inhibitors decreased the H₂O₂ stimulation of ERK and JNK show that oxidized LDL increases ERK activation. DHA modulates ERK1/2 signaling. It is of interest to note that a subunit of PI3K which is upstream of ERK, PIK3CG has been associated to Autism {32647}.

ERK2 also phosphorylates & activates PLA2s, both calcium-dependent and calcium-independent and functions similarly to ERK1.

ASK1 binds to GSTM1 and is inhibited by GSTM1, TXN and GLRX1. GSTM1 is associated with autism. ASK1 is important in MAPK activation due to TNFa exposure.

MEKK1 binds to and regulates GSTM1, associated with autism. MEKK1 overexpression upregulates COX-2 through JNK and p38 activation.

Thioredoxin (TXN or TRX) is a small cytosolic enzyme with oxidoreductase activity that contains a dithiol-disulfide active site. It contributes to maintaining protein stability under conditions of oxidative stress. TXN binds to the N-terminal region of ASK1 in a fashion highly dependent on the redox status of TRX. When TXN is expressed, ASK1 kinase activity and ASK1-dependent apoptosis are inhibited. Thus, when reduced TXN binds to ASK1, ASK1 is inactive but when TXN is oxidized, the complex breaks up and ASK1 returns to activity. TXN2 is a mitochondrial form coded for by a different gene.

Glutaredoxin (GLRX, GRX or thioltransferase) is a small cytosolic enzyme that catalyzes GSSG oxidoreduction reactions in the presence of glutathione reductase (discussed above) and NADPH; it also acts as a GSH-dependent hydrogen donor for ribonucleotide reductase. Like TRX, GLRX may be a sensor molecule that recognizes oxidative stress. Like TXN, GLRX binds to ASK1 but to the C-terminal region instead. GLRX inhibits ASK1 when it is bound but when released allows ASK1 activation. GLRX and TXN are released from ASK1 by different mechanisms as is GSTM1 by still a third mechanism. However, all three participate in ASK1 regulation.

HSP 72, binds to and inhibits JNK

GSTP1 binds to and regulates JNK, a key MAPK. GSTP1 was associated with autism. Other proteins besides GSTP1, e.g. HSP72 (heat shock protein 72), and EVI1 (ecotropic viral integration site-1) also bind to JNK, inhibit its function and thus regulate it. GSTP1 binds to JNK1 (MAPK8) and may also bind as well to JNK2 (MAPK9) and JNK3 (MAPK10), which are distinct but closely related genes. Extensive studies, discussed earlier, document GSTP1 binding to JNK without specifying the specific form. Binding to JNK1 has also been documented; since almost complete sequence homology between JNK1, JNK2, and JNK3 for a putative GSTP1-binding site has been demonstrate, it seems likely that GSTP1 may bind to and regulates all three. The binding of HSP72 and EVI1 to specific forms of JNK has not been studied. JNK1 and JNK2 are ubiquitous but JNK3 is brain-specific.

GSTM1, is one of the proteins that bind to and regulate ASK1 and MEKK1. GSTM1 is associated with autism. GSTM1 is an important contributor to controlling oxidative stress through conjugation of xenobiotics for their detoxification. We recently reported it to be associated with autism.

MKP1 is one of the Dual specific phosphatases and inhibits JNK, ERK and p38 and may also inhibit upstream MAPKs. MKP1 is involved in dynamic regulation of both pro- and anti-inflammatory cytokines by in innate immune responses. MKP1 dephosphorylates MAPK's and is regulated by MAPK's.

### Upstream Mediators of MAPK Activation

Proteins of two genes associated with autism, RELN (reelin protein) and APOE (apolipoprotein E protein), competitively bind the same apolipoprotein E receptor, APOER2, a transmembrane protein expressed during development especially in brain, particularly in neurons and cells that are components of the blood brain barrier. APOE binding to APOER2 decreases the activation of JNK through the APOER2 receptor. The allele associated with autism has a lower binding affinity for APOER2. Low levels of reelin protein in blood and brain were reported in individuals with autism. Both association and lack of association with autism has been reported for various RELN polymorphisms. The 5′ trinucleotide repeat of RELN has repeatedly been found to be associated with autism. The longer repeat allele, associated with autism, correlates with slower reelin protein synthesis.

On the cytoplasmic side of the cell membrane, APOER2 recruits and binds the JIP1 and JIP2 (JNK interacting proteins), members of the JIP group of scaffolding proteins for the JNK-signaling pathway. APOER2 binds both JIP-1 and JIP-2 through a proline-rich domain. Interestingly, the occurrence of the splice variant of APOER2 containing this proline-rich domain and the expression of JIP-2 coincide during the period of brain development when neurons differentiate. This makes JIP2 of particular interest for autism. Stockinger et al. found evidence that the MAPK proteins, MLK3 (MAP3K11) and MKK7 (MAP2K7), are recruited to and bound to JIP-2 along with JNK as part of an APOER2 multicomponent signaling pathway. They interpreted their results to demonstrate a molecular link between APOER2 and the JNK signaling pathway. APOER2 also bind to PSD95, a multidomain scaffolding protein, through the PDZ1 domain of PSD95, a domain that can mediate interactions with other proteins. PSD95 may recruit proteins to post-synaptic sites on neurons. PSD95 recruits neuroligands 1, 3 & 4 but not neuroligand 2. Interestingly neuroligands 1, 3 & 4 but not neuroligand 2 are associated with autism. Neuroligands are a family of postsynaptic transmembrane proteins on dendrites that associate with presynaptic partners, the beta-neurexins. PSD95 also binds to GLUR6, a protein that is associated with autism and that contributes to JNK activation. PSD95 also binds to the NMDA receptor subunit, NR2A (associated with autism), and to NR2B through its PDZ2 domain.

### Protein Kinases C & A

Protein kinases C and A activate MAPKs that themselves contribute to upstream activation of PLA2s, LOs and COXs and may be upstream of the NF-kB pathway. Protein kinases C and A also directly activate PLA2s PLA2s. Regarding nomenclature: PKA protein=PRKA gene, R=regulatory subunit, C=catalytic subunit. PRKCB1 (is associated with autism). Eleven PKCs and PKAs are included in the project and their rationales are quite similar. PRKAR1A, PRKAR1B, PRKAR2A, PRKAR2B, PRKACB, PRKACG, PRKCA, PRKCB2.

PLCG1 activates both PKAs and PKCs and is an important upstream mediator of MAPK activation of PLA2s.

### NFkB Pathway

The NFkB pathway is important for the activation of PLA2, COX-2 and LO's.

TABLE 3
Genes related control of DHA/AA/EPA metabolism
MAPK

| | |
|---|---|
| THIOREDOXIN | TXN |
| THIOREDOXIN REDUCTASE 1 | TXNRD1 |
| GLUTAREDOXIN | GLRX |
| HEAT-SHOCK 70-KD PROTEIN 1A (HSP72) | HSPA1A |
| POSTSYNAPTIC DENSITY 95 | PSD 95 |
| LOW DENSITY LIPOPROTEIN RECEPTOR-RELATED PROTEIN 8 (APOER2) | LRP8 |
| LOW DENSITY LIPOPROTEIN RECEPTOR | LDLR |
| DISABLED, DROSOPHILA, HOMOLOG OF, 1 | DAB1 |
| C-JUN KINASE 1 (JNK1) | MAPK8 |
| C-JUN KINASE 2 (JNK2) | MAPK9 |
| C-JUN KINASE 3 (JNK3) | MAPK10 |
| APOPTOSIS SIGNAL-REGULATING KINASE 1; ASK1 (ASK1) | MAP3K5 |
| MAP/ERK KINASE KINASE 1 (MEKK1) | MAP3K1 |
| MITOGEN-ACTIVATED PROTEIN KINASE KINASE 6 (MKK6) (MAPKK6) (MEK6) | MAP2K6 |
| MITOGEN-ACTIVATED PROTEIN KINASE KINASE 3 (MKK3) (MAPKK3) (MEK3) | MAP2K3 |
| MITOGEN-ACTIVATED PROTEIN KINASE KINASE KINASE 11 (MLK3) | MAP3K11 |
| MITOGEN-ACTIVATED PROTEIN KINASE 14 (P38) | MAPK14 |
| MITOGEN-ACTIVATED PROTEIN KINASE 3 (ERK1) | MAPK3 |
| MITOGEN-ACTIVATED PROTEIN KINASE 1; (ERK2) | MAPK1 |
| MITOGEN-ACTIVATED KINASE KINASE KINASE 1 (MEKK1) | MAP3K1 |
| NUCLEAR RECEPTOR SUBFAMILY 2, GROUP C, MEMBER 2 (TAK1) | NR2C2 |
| MITOGEN-ACTIVATED PROTEIN KINASE KINASE KINASE 2 (MEKK2) | MAP3K2 |
| MITOGEN-ACTIVATED PROTEIN KINASE KINASE KINASE 4 (MEKK4) | MAP3K4 |
| JNK-ACTIVATING KINASE 2 (MAP2K7) (MKK7) (MEK7) | JNKK2 |
| JNK-ACTIVATED KINASE 1 (MAP2K4) (MKK4) (MEK4) | JNKK1 |
| MAP KINASE PHOSPHATASE 5 (DUSP10) | MKP |
| MAP KINASE PHOSPHATASE 1 (DUSP1) | MKP1 |
| MAP KINASE PHOSPHATASE 6 (DUSP14) | MKP6 |
| MAP KINASE PHOSPHATASE 7 (DUSP16) | MKP7 |
| MAP KINASE PHOSPHATASE X (DUSP7) | MKPX |
| MAP KINASE PHOSPHATASE 3 (DUSP6) | MKP3 |
| MAP KINASE PHOSPHATASE 4 (DUSP9) | MKP4 |
| JNK-INTERACTING PROTEIN 1 | JIP1 |
| JNK-INTERACTING PROTEIN 2 | JIP2 |
| JNK-INTERACTING PROTEIN 3 (JSAP1) | JIP3 |
| JNK-INTERACTING PROTEIN 4 | JIP4 |
| PROTEIN PHOSPHATASE 2, CATALYTIC SUBUNIT, ALPHA ISOFORM | PPP2CA |
| CYCLIN-DEPENDENT KINASE INHIBITOR 1A (p21cip) | CDKN1A |
| MADS BOX TRANSCRIPTION ENHANCER FACTOR 2, POLYPEPTIDE C | MEF2C |
| V-JUN AVIAN SARCOMA VIRUS 17 ONCOGENE HOMOLOG | JUN |
| ONCOGENE JUN-D | JUND |
| V-FOS FBJ MURINE OSTEOSARCOMA VIRAL ONCOGENE HOMOLOG B | FOSB |
| V-FOS FBJ MURINE OSTEOSARCOMA VIRAL ONCOGENE HOMOLOG | FOS |
| NUCLEAR FACTOR ERYTHROID 2-LIKE 2 (NRF2) | NFE2L2 |
| V-MAF AVIAN MUSCULOAPONEUROTIC FIBROSARCOMA ONCOGENE HOMOLOG | MAF |
| NADPH-DEPENDENT DIFLAVIN OXIDOREDUCTASE 1 | NDOR1 |
| FAS LIGAND | FASL |
| FAS ANTIGEN | FAS |
| TUMOR PROTEIN p53 | TP53 |
| ECOTROPIC VIRAL INTEGRATION SITE 1 | EVI1 |
| CYCLIN-DEPENDENT KINASE INHIBITOR 2D | CDKN2D |

NFKappa B
The NFKappa B system is important in DHA/AA/EPA metabolism.
IKK

| | |
|---|---|
| INHIBITOR OF KAPPA LIGHT CHAIN GENE ENHANCER IN B CELLS, KINASE OF, BETA | IKBKB |
| INHIBITOR OF KAPPA LIGHT POLYPEPTIDE GENE ENHANCER IN B CELLS, KINASE OF, GAMMA | IKBKG |
| INHIBITOR OF KAPPA LIGHT POLYPEPTIDE GENE ENHANCER IN B CELLS, KINASE | IKBKAP |
| COMPLEX-ASS.OCIATED PROTEIN | |
| INHIBITOR OF KAPPA LIGHT POLYPEPTIDE GENE ENHANCER IN B CELLS, KINASE OF, EPSILON | IKBKE |
| I-KAPPA-B KINASE-INTERACTING PROTEIN | IKIP |
| NUCLEAR FACTOR KAPPA-B, SUBUNIT 1 | NFKB1 |
| V-REL AVIAN RETICULOENDOTHELIOSIS VIRAL ONCOGENE HOMOLOG A | RELA |
| NUCLEAR FACTOR KAPPA-B, SUBUNIT 2 | NFKB2 |
| NFKB-REPRESSING FACTOR | NRF |
| CONSERVED HELIX-LOOP-HELIX UBIQUITOUS KINASE | CHUK |
| V-REL AVIAN RETICULOENDOTHELIOSIS VIRAL ONCOGENE HOMOLOG B | RELB |
| V-REL AVIAN RETICULOENDOTHELIOSIS VIRAL ONCOGENE HOMOLOG | REL |
| NUCLEAR FACTOR OF KAPPA LIGHT CHAIN GENE ENHANCER IN B CELLS INHIBITOR, ALPHA | NFKBIA |
| NUCLEAR FACTOR OF KAPPA LIGHT CHAIN GENE ENHANCER IN B CELLS INHIBITOR, BETA | NFKBIB |
| INHIBITOR OF KAPPA LIGHT POLYPEPTIDE GENE ENHANCER IN B CELLS, KINASE OF, ALPHA | IKBKA |
| INHIBITOR OF KAPPA LIGHT CHAIN GENE ENHANCER IN B CELLS, KINASE OF, BETA; | IKBKB |
| INHIBITOR OF KAPPA LIGHT POLYPEPTIDE GENE ENHANCER IN B CELLS, KINASE OF, GAMMA | IKBKG |
| V-AKT MURINE THYMOMA VIRAL ONCOGENE HOMOLOG 1; | AKT1 |
| V-AKT MURINE THYMOMA VIRAL ONCOGENE HOMOLOG 2; | AKT2 |
| I-KAPPA-B-INTERACTING RAS-LIKE PROTEIN 1 | KAPPA-B-RAS1 |
| I-KAPPA-B-INTERACTING RAS-LIKE PROTEIN 2 | KAPPA-B-RAS2 |

PKC/PKA
PK C's and PKA's are important for both MAPK and NFkB activation.
They are also important in direct PLA2 activation.

| | |
|---|---|
| PROTEIN KINASE C alpha | PKCA |
| PROTEIN KINASE C beta | PKCB1 |
| PROTEIN KINASE C beta2 | PKCB2 |
| PROTEIN KINASE C zeta | PKCZ |
| PROTEIN KINASE C theta | PKCT |
| PROTEIN KINASE C Iota | PRKCI |
| PROTEIN KINASE C Delta | PRKCD |
| PROTEIN KINASE A alpha | PRKAR1A |
| PROTEIN KINASE A beta | PRKAR1B |
| PROTEIN KINASE A | PRKAR2A |
| PROTEIN KINASE A | PRKAR2B |
| PROTEIN KINASE A | PRKACB |
| PROTEIN KINASE A | PRKACG |

Upstream activators of MAPK, NFkB, PKA and PKC.

| | |
|---|---|
| TUMOR NECROSIS FACTOR alpha | TNFA |
| TNF RECEPTOR-ASSOCIATED FACTOR 2 | TRAF2 |
| TUMOR NECROSIS FACTOR RECEPTOR SUPERFAMILY, MEMBER 1A (TNFR1) | TNFRSF1A |
| LIPOPOLYSACCHARIDE-BINDING PROTEIN | LBP |
| TUMOR NECROSIS FACTOR RECEPTOR 1-ASSOCIATED DEATH DOMAIN PROTEIN | TRADD |
| Toll like receptor 4 | TLR4 |
| Toll like receptor 2 | TLR2 |
| INTERLEUKIN 1 RECEPTOR, TYPE II | IL1R2 |
| INTERLEUKIN 1 RECEPTOR, TYPE I | IL1R1 |
| TRANSFORMING GROWTH FACTOR, BETA-1 | TGFB1 |
| SIGNAL TRANSDUCER AND ACTIVATOR OF TRANSCRIPTION 6 | STAT6 |
| INTERLEUKIN 1-BETA | IL1B |

III. Ongoing Gene Expression and/or Expressed Protein Activity Analysis

Each, any or a combination of the above stress and lipid relevant genes may be further assessed or monitored for expression, being linked to altered levels of oxidative stress and/or lipid metabolites. Methods known and recognized in the art may be utilized to assay and test RNA, protein levels, and enzyme activity, including phosphorylation of or by kinases, and downstream modulation via signaling molecules.

### IV. Disease-Relevant Genes

As a complement or corollary to the genetic and biochemical markers of oxidative stress and lipid metabolism, genes associated with stress-relevant diseases may also or further be assessed. This provides a more significant assessment of risk or disease, particularly in view of the fact that complex diseases are caused by a combination of multiple genetic and environmental components. Thus, a complete scan incorporating genetically-associated disease related markers and some candidate genes provides disease relevant analysis and outcomes. Exemplary disease associated genes and markers are provided below. In particular, corollary genes associated with autism, asthma, Alzheimer's disease are provided.

### Autism:

While not apparent, many of the genes that have previously been associated with autism are related to each other and to the activation/inhibition/control of MAPK/NFkB/PKCPKA. Many are therefore relevant to the metabolism and regulation of DHA, AA, Lipoxins and Resolvins.

The following provides autism specific or relevant genes, which are identified as associated with Autism.

TABLE 4
AUTISM

| | |
|---|---|
| ADENOSINE DEAMINASE | ADA |
| APOLIPOPROTEIN E | APOE |
| COMPLEMENT COMPONENT 4B | C4B |
| ENGRAILED 2 | EN2 |
| FORKHEAD BOX P2 | FOXP2 |
| GAMMA-AMINOBUTYRIC ACID RECEPTOR, BETA-1 | GABRB1 |
| GAMMA-AM1NOBUTYRIC ACID RECEPTOR, ALPHA-4 | GABRA4 |
| GAMMA-AMINOBUTYRIC ACID RECEPTOR, BETA-3 | GABRB3 |
| GLUTAMATE RECEPTOR, IONOTROPIC, KAINATE 2 (GluR6) | GRIK2 |
| GLUTAMATE RECEPTOR, METABOTROPIC, 8 | GRM8 |
| MONOAMINE OXIDASE A | MAOA |
| METHYL-CpG-BINDING PROTEIN 2 (Rett syndrome) | MECP2 |
| OXYTOCIN RECEPTOR | OXTR |
| PARAOXONASE 1 | PON1 |
| PROTEIN KINASE C, BETA-1 | PRKCB1 |
| REELIN | RELN |
| SOLUTE CARRIER FAMILY 25 (MITOCHONDRIAL CARRIER, ARALAR), MEMBER 12 | SLC25A12 |
| SOLUTE CARRIER FAMILY 6 (NEUROTRANSMITTER TRANSPORTER, SEROTONIN), MEMBER 4 | SLC6A4 |
| TRYPTOPHAN 2,3-DIOXYGENASE | TDO2 |
| TRYPTOPHAN HYDROXYLASE 2 | TPH2 |
| UBIQUITIN-CONJUGATING ENZYME E2H | UBE2H |
| FRAGILE SITE MENTAL RETARDATION 1 GENE (FRAXAX) | FMR1 |
| WINGLESS-TYPE MMTV INTEGRATION SITE FAMILY, MEMBER 2 | WNT2 |
| TSC1 GENE (HAMARTIN) | TSC1 |
| TSC2 GENE (TUBERIN) | TSC2 |
| SOLUTE CARRIER FAMILY 40 (IRON-REGULATED TRANSPORTER), MEMBER 1 | SLC40A1 |
| METAL-REGULATORY TRANSCRIPTION FACTOR 1 | MTF1 |
| MAJOR HISTOCOMPATIBILITY COMPLEX, CLASS I, A | HLA-A |
| HOMEOBOX B1 | HOXB1 |
| V-HA-RAS HARVEY RAT SARCOMA VIRAL ONCOGENE HOMOLOG | HRAS |
| INOSITOL POLYPHOSPHATE-1-PHOSPHATASE | INPP1 |
| LAMININ, BETA-1 | LAMB1 |
| NEUROFIBROMATOSIS, TYPE I | NF1 |
| NEUROLIGIN 1 | NLGN1 |
| NEUROLIGIN 3 | NLGN3 |
| NEUROLIGIN 4, Y-LINKED | NLGN4Y |
| PHOSPHATIDYLINOSITOL 3-KINASE, CATALYTIC, GAMMA | PIK3CG |
| MET PROTOONCOGENE | MET |
| 5,10-METHYLENETETRAHYDROFOLATE REDUCTASE; | MTHFR |
| REDUCED FOLATE CARRIER 1; | RFC1 |
| TRANSCOBALAMIN II DEFICIENCY | TCN2 |
| METHIONINE SYNTHASE REDUCTASE; | MTRR |
| GLUTATHIONE S-TRANSFERASE, MU-1 | GSTM1 |
| GLUTATHIONE S-TRANSFERASE, PI | GSTP1 |
| DIHYDROFOLATE REDUCTASE | DHFR |

PSD95 related genes.

PSD95 Chr17: 7063754 . . . 7033933
NNOS Chr12: 116283965 . . . 116135362
CAPON chr1: 160306205 . . . 160604864
DAB1 Chr1: 58488799 . . . 57236167
APOER2 Chr1: 53566409 . . . 53483800
VLDLRChr9: 2611793 . . . 2644485
GRIN2B Chr12: 14024319 . . . 13605411
NRP1 (Neuropilin1)
SHANK1
SHANK2
HOMER1
HOMER2
HOMER3
CAMK2A
DCC
EDN1
EDNRB
ESR1
Esr2
Gkap
GNA12
GNA13
LDLR
NRP2
NTN1
NTN2L
NTN4
SEMA3B
SEMA3C
SEMA3E
SEMA3F
SEMA4A
SEMA4C
SEMA4D
SEMA4F
SEMA5A
SEMA5B
SEMA6A
SEMA7A
STAT6
TANK
TGFB1
tiam1
zip70
LRP8 (APOER2)
Sema3A
PTEN
GRIP1
PDZK1
CAPN9
NTNG1
GRM2
DLG1
CAMK2N2
AMPA 2
CAMK2B
CAMK2G
DLG5
DBN1
CNTNAP2

Cell cycle relevant genes

PIK3CG
AKT1 (alpha):
AKT2 (beta):
AKT3 (gamma):
PDK1:
TSC2:
NF
Rheb2:
mTOR (FRAP1):
S6K1 (RPS6KB1):
S6K2 (RPS6KB2):
4E-BP1 (EIF4EBP1):
4E-BP2 (EIF4EBP2):
p27kip1 (CDKN1B):
CDK2:
14-3-3 sigma (stratifin; SFN):
14-3-3 epsilon (YWHAE):
14-3-3 zeta (YWHAZ):

Genes that may facilitate extra brain growth via MAPK and or DHA or by limiting nucleotides for DNA synthesis.

| | |
|---|---|
| CYCLIN-DEPENDENT KINASE INHIBITOR 1B (p27(KIP1)) | CDKN1B |
| PHOSPHATASE AND TENSIN HOMOLOG | PTEN |
| FKBP12-RAPAMYCIN COMPLEX-ASSOCIATED PROTEIN 1; (MTOR) | FRAP1 |
| CYCLIN-DEPENDENT KINASE INHIBITOR 2A | CDKN2A |
| METHYLENETETRAHYDROFOLATE DEHYDROGENASE 1 | MTHFD1 |
| 5-α-METHYLTETRAHYDROFOLATE-HOMOCYSTEINE S-METHYLTRANSFERASE | MTR |
| THYMIDYLATE SYNTHETASE | TYMS |
| DNA METHYLTRANSFERASE 1 | DNMT1 |
| CATECHOL-O-METHYLTRANSFERASE | COMT |
| RAS-ASSOCIATED PROTEIN RAB3A | RAB3A |
| CYCLIN D1 | CCND1 |
| ENDOTHELIN 1 (ET1) | EDN1 |
| ENDOTHELIN RECEPTOR, TYPE B (ETB) | EDNRB |
| GUANINE NUCLEOTIDE-BINDING PROTEIN, ALPHA-12 (Galpha12) | GNA12 |
| GUANINE NUCLEOTIDE-BINDING PROTEIN, ALPHA-13 (Galpha13) | GNA13 |
| MITOGEN-ACTIVATED PROTEIN KINASE KINASE KINASE 12 (Muk) | MAP3K12 |
| T-CELL LYMPHOMA INVASION AND METASTASIS 1 | TIAM1 |
| RHO FAMILY, SMALL GTP-BINDING PROTEIN RAC1 | RAC1 |
| V-RAF-1 MURINE LEUKEMIA VIRAL ONCOGENE HOMOLOG 1 | RAF1 |

Others

| | |
|---|---|
| HEME OXYGENASE 1 | HMOX1 |
| HEME OXYGENASE 2 | HMOX2 |
| MONOCYTE DIFFERENTIATION ANTIGEN CD14 | CD14 |
| CD40 ANTIGEN | CD40 |
| COMPLEMENT COMPONENT 4-BINDING PROTEIN, ALPHA | C4BPA |
| NEUREXIN 1 | NRXN1 |
| NEUREXIN 2 | NRXN2 |
| NEUREXIN 3 | NRXN3 |
| GROWTH ARREST- AND DNA DAMAGE-INDUCIBLE GENE GADD45, BETA | GADD45B |
| INHIBITOR OF APOPTOSIS, X-LINKED | XIAP |
| KERATINOCYTE GROWTH FACTOR | KGF |
| COLONY-STIMULATING FACTOR 2 RECEPTOR, ALPHA | CSF2RA |
| A DISINTEGRIN AND METALLOPROTEINASE DOMAIN 33 | ADAM33 |
| SECRETOGLOBIN, FAMILY 1A, MEMBER 1 | SCGB1A1 |
| MUCIN 7, SALIVARY | MUC7 |
| IKK COMPLEX-ASSOCIATED PROTEIN | IKAP |
| CYSTEINYL LEUKOTRIENE RECEPTOR 2 | CYSLTR2 |

PSD95 genes (synapse integrity, relevant in other neurodevelopmental disorders) In addition as we spoke of many of these are important for synapse integrity which is modified by lipid content in the brain. In fact, some are directly related to lipid binding (APOE which is associated with Autism and alzheimers, APOER2, VLDLDR) Others are related to membrane and oxidative stress such as NNOS and CAPON.

### Asthma:

The following provides asthma specific or relevant genes, which are identified as associated with asthma.

TABLE 5
Asthma Relevant Genes

| | |
|---|---|
| ABO | ABO BLOOD GROUP |
| ADA | ADENOSINE DEAMINASE |
| ADAM33 | A DISINTEGRIN AND METALLOPROTEINASE DOMAIN 33 |
| ADCY9 | ADENYLATE CYCLASE 9 |
| ADRB2 | BETA-2-ADRENERGIC RECEPTOR |
| AICDA | ACTIVATION-INDUCED CYTIDINE DEAMINASE |
| ALOX5 | ARACHIDONATE 5-LIPOXYGENASE |
| ALOX5AP | ARACHIDONATE 5-LIPOXYGENASE-ACTIVATING PROTEIN |
| AOAH | ACYLOXYACYL HYDROLASE |
| BAT1 | HLA-B-ASSOCIATED TRANSCRIPT 1 |
| BDKRB2 | BRADYKININ RECEPTOR B2 |
| C3 | COMPLEMENT COMPONENT 3 |
| C3AR1 | COMPLEMENT COMPONENT 3a RECEPTOR 1 |
| C5 | COMPLEMENT COMPONENT 5 |
| C5orf20 | DENDRITIC CELL NUCLEAR PROTEIN 1 |
| CARD15 | NUCLEOTIDE-BINDING OLIGOMERIZATION DOMAIN PROTEIN 2 |
| CAT | CATALASE |
| CCL11 | CHEMOKINE, CC MOTIF, LIGAND 11 |
| CCL2 | CHEMOKINE, CC MOTIF, LIGAND 2 |
| CCL24 | CHEMOKINE, CC MOTIF, LIGAND 24 |
| CCL26 | CHEMOKINE, CC MOTIF, LIGAND 26 |
| CCL5 | CHEMOKINE, CC MOTIF, LIGAND 5 |
| CCR3 | CHEMOKINE, CC MOTIF, RECEPTOR 3 |
| CCR5 | CHEMOKINE, CC MOTIF, RECEPTOR 5 |
| CD14 | MONOCYTE DIFFERENTIATION ANTIGEN CD14 |
| CD40 | CD40 ANTIGEN |
| CD86 | CD86 ANTIGEN |
| CFTR | CYSTIC FIBROSIS TRANSMEMBRANE CONDUCTANCE REGULATOR |
| CHRM1 | CHOLINERGIC RECEPTOR, MUSCARINIC, 1 |
| CHRM2 | CHOLINERGIC RECEPTOR, MUSCARINIC, 2 |
| CHRM3 | CHOLINERGIC RECEPTOR, MUSCARINIC, 3 |
| CLCA1 | CHLORIDE CHANNEL, CALCIUM-ACTIVATED, 1 |
| CMA1 | CHYMASE 1 |
| CRHR1 | CORTICOTROPIN-RELEASING HORMONE RECEPTOR 1 |
| CSF2 | COLONY-STIMULATING FACTOR 2 |
| CTLA4 | CYTOTOXIC T LYMPHOCYTE-ASSOCIATED 4 |
| CX3CL1 | CHEMOKINE, CX3C MOTIF, LIGAND 1 |
| CXCR3 | CHEMOKINE, CXC MOTIF, RECEPTOR 3 |
| CYP1A1 | CYTOCHROME P450, SUBFAMILY I, POLYPEPTIDE 1 |
| CYP2J2 | CYTOCHROME P450, SUBFAMILY IIJ, POLYPEPTIDE 2 |
| CYSLTR1 | CYSTEINYL LEUKOTRIENE RECEPTOR 1 |
| CYSLTR2 | CYSTEINYL LEUKOTRIENE RECEPTOR 2 |
| DAP3 | DEATH-ASSOCIATED PROTEIN 3 |
| DEFB1 | DEFENSIN, BETA, 1 |
| EDN1 | ENDOTHELIN 1 |
| EGFR | EPIDERMAL GROWTH FACTOR RECEPTOR |
| FCGR1A | Fc FRAGMENT OF IgG, HIGH AFFINITY Ia, RECEPTOR FOR |
| FCGR1B | Fc FRAGMENT OF IgG, HIGH AFFINITY Ib, RECEPTOR FOR |
| FCGR2A | Fc FRAGMENT OF IgG, LOW AFFINITY IIa, RECEPTOR FOR |
| FLG | FILAGGRIN |
| FUT2 | FUCOSYLTRANSFERASE 2 |
| FUT3 | FUCOSYLTRANSFERASE 3 |
| GATA3 | GATA-BINDING PROTEIN 3 |
| GNB1 | GUANINE NUCLEOTIDE-BINDING PROTEIN, BETA-1 |
| GPR44 | G PROTEIN-COUPLED RECEPTOR 44 |
| GSTM1 | GLUTATHIONE S-TRANSFERASE, MU-1 |
| GSTM3 | GLUTATHIONE S-TRANSFERASE, MU-3 |
| GSTP1 | GLUTATHIONE S-TRANSFERASE, PI |
| GSTT1 | GLUTATHIONE S-TRANSFERASE, THETA-1 |
| HAVCR1 | HEPATITIS A VIRUS CELLULAR RECEPTOR 1 |
| HAVCR2 | HEPATITIS A VIRUS CELLULAR RECEPTOR 2 |
| HLA-DPB1 | MAJOR HISTOCOMPATIBILITY COMPLEX, CLASS II, DP BETA-1 |
| HLA-DQA1 | MAJOR HISTOCOMPATIBILITY COMPLEX, CLASS II, DQ ALPHA-1 |
| HLA-DQB1 | MAJOR HISTOCOMPATIBILITY COMPLEX, CLASS II, DQ BETA-1 |
| HLA-DRB1 | MAJOR HISTOCOMPATIBILITY COMPLEX, CLASS II, DR BETA-1 |
| HNMT | HISTAMINE N-METHYLTRANSFERASE |
| IFNG | INTERFERON, GAMMA |
| IFNGR1 | INTERFERON, GAMMA, RECEPTOR 1 |
| IKBKAP | INHIBITOR OF KAPPA LIGHT POLYPEPTIDE GENE ENHANCER IN B |
| | CELLS, KINASE COMPLEX-ASSOCIATED PROTEIN |
| IL10 | INTERLEUKIN 10 |
| IL12B | INTERLEUKIN 12B |
| IL13 | INTERLEUKIN 13 |
| IL13RA1 | INTERLEUKIN 13 RECEPTOR, ALPHA-1 |
| IL15 | INTERLEUKIN 15 |
| IL16 | INTERLEUKIN 16 |
| IL17F | INTERLEUKIN 17F |
| IL18 | INTERLEUKIN 8 |
| IL1B | INTERLEUKIN 1B |
| IL1RA | INTERLEUKIN 1 RECEPTOR ANTAGONIST |
| IL3 | INTERLEUKIN 3 |
| IL4 | INTERLEUKIN 4 |
| IL4R | INTERLEUKIN 4 RECEPTOR |
| IL5 | INTERLEUKIN 5 |
| IL8 | INTERLEUKIN 8 |
| IL8RA | INTERLEUKIN 8 RECEPTOR, ALPHA |
| IL9 | INTERLEUKIN 9 |
| IRF1 | INTERFERON REGULATORY FACTOR 1 |
| ITGB3 | INTEGRIN, BETA-3 |
| JUND | ONCOGENE JUN-D |
| KDR | KINASE INSERT DOMAIN RECEPTOR |
| LELP1 | LATE CORNIFIED ENVELOPE-LIKE PROLINE-RICH 1 |
| LTA | LYMPHOTOXIN-ALPHA |
| LTA4H | LEUKOTRIENE A4 HYDROLASE |
| LTC4S | LEUKOTRIENE C4 SYNTHASE |
| MIF | MACROPHAGE MIGRATION INHIBITORY FACTOR |
| MMP1 | MATRIX METALLOPROTEINASE 1 |
| MMP9 | MATRIX METALLOPROTEINASE 9 |
| MPO | MYELOPEROXIDASE |
| MS4A2 | MEMBRANE-SPANNING 4 DOMAINS, SUBFAMILY A, MEMBER 2 |
| MUC2 | MUCIN 2, INTESTINAL |
| MUC7 | MUCIN 7, INTESTINAL |
| MYLK | MYOSIN LIGHT CHAIN KINASE |
| NAT1 | N-ACETYLTRANSFERASE 1 |
| NAT2 | N-ACETYLTRANSFERASE 2 |
| NOD1 | NUCLEOTIDE-BINDING OLIGOMERIZATION DOMAIN PROTEIN 1 |
| NOS1 | NITRIC OXIDE SYNTHASE 1 |
| NOS2A | NITRIC OXIDE SYNTHASE 2A |
| NOS3 | NITRIC OXIDE SYNTHASE 3 |
| NQO1 | NAD(P)H DEHYDROGENASE, QUINONE 1 |
| PLA2G7 | PHOSPHOLIPASE A2, GROUP VII |
| PDGFRA | PLATELET-DERIVED GROWTH FACTOR RECEPTOR, ALPHA |
| PGDS | PROSTAGLANDIN D2 SYNTHASE, HEMATOPOIETIC |
| PHF11 | PHD FINGER PROTEIN 11 |
| PTGDR | PROSTAGLANDIN D2 RECEPTOR |
| PTGER2 | PROSTAGLANDIN E RECEPTOR 2, EP2 SUBTYPE |
| PTGER3 | PROSTAGLANDIN E RECEPTOR 3, EP3 SUBTYPE |
| PTGER4 | PROSTAGLANDIN E RECEPTOR 4, EP4 SUBTYPE |
| PTGIR | PROSTAGLANDIN 12 RECEPTO |
| PTPN22 | PROTEIN TYROSINE PHOSPHATASE, NONRECEPTOR-TYPE, 22 |
| CCL5 | CHEMOKINE, CC MOTIF, LIGAND 5 |
| RNASE3 | RIBONUCLEASE A FAMILY, 3 |
| RUNX1 | RUNT-RELATED TRANSCRIPTION FACTOR |
| SCGB1A1 | UTEROGLOBI |
| SCGB3A2 | SECRETOGLOBIN, FAMILY 3A, MEMBER 2 |
| SERPINA3 | ALPHA-1-ANTICHYMOTRYPSI |
| SERPINE1 | PLASMINOGEN ACTIVATOR INHIBITOR |
| SFTPC | SURFACTANT, PULMONARY-ASSOCIATED PROTEIN C |
| SPINK5 | SERINE PROTEASE INHIBITOR, KAZAL-TYPE, 5 |
| SPP1 | SECRETED PHOSPHOPROTEIN |
| STAT3 | SIGNAL TRANSDUCER AND ACTIVATOR OF TRANSCRIPTION |
| STAT4 | SIGNAL TRANSDUCER AND ACTIVATOR OF TRANSCRIPTION |
| STAT6 | SIGNAL TRANSDUCER AND ACTIVATOR OF TRANSCRIPTION |
| TAP1 | TRANSPORTER, ATP-BINDING CASSETTE, MAJOR HISTOCOMPATIBILITY |
| | COMPLEX, 1 |
| TBX21 | T-BOX 2 |
| TBXA2R | THROMBOXANE A2 RECEPTOR, PLATELET |
| TGFB1 | TRANSFORMING GROWTH FACTOR, BETA-1 |
| TIMD4 | T-CELL IMMUNOGLOBULIN AND MUCIN DOMAINS-CONTAINING PROTEIN |
| TIMELESS | TIMELESS, DROSOPHILA, HOMOLOG OF |
| TIMP1 | TISSUE INHIBITOR OF METALLOPROTEINASE |
| TLR10 | TOLL-LIKE RECEPTOR 1 |
| TLR2 | TOLL-LIKE RECEPTOR |
| TLR4 | TOLL-LIKE RECEPTOR |
| TLR6 | TOLL-LIKE RECEPTOR |
| TLR9 | TOLL-LIKE RECEPTOR |
| TNC | TENASCIN |
| TNF | TUMOR NECROSIS FACTOR |

### Alzheimers:

The following provides Alzheimer's disease specific or relevant genes, which are identified as associated with Alzheimer's.

TABLE 6
Alzheimers Disease Relevant Genes

| | |
|---|---|
| A2M | ALPHA-2-MACROGLOBULIN |
| ABCA2 | ATP-BINDING CASSETTE, SUBFAMILY A, MEMBER 2 |
| ABCA1 | ATP-BINDING CASSETTE, SUBFAMILY A, MEMBER 1 |
| ABCA12 | ATP-BINDING CASSETTE, SUBFAMILY A, MEMBER 12 |
| ACE | ANGIOTENSIN I-CONVERTING ENZYME |
| AHSG | ALPHA-2-HS-GLYCOPROTEIN |
| APBB1 | AMYLOID BETA A4 PRECURSOR PROTEIN-BINDING, FAMILY B, |
| | MEMBER 1 |
| APBB2 | AMYLOID BETA A4 PRECURSOR PROTEIN-BINDING, FAMILY B, |
| | MEMBER 2 |
| APH1B | ANTERIOR PHARYNX DEFECTIVE 1, C. ELEGANS, HOMOLOG OF, B |
| APOA1 | APOLIPOPROTEIN A-I |
| APOC1 | APOLIPOPROTEIN C-I |
| APOC3 | APOLIPOPROTEIN C-III |
| APOD | APOLIPOPROTEIN D |
| APOER2 | LOW DENSITY LIPOPROTEIN RECEPTOR-RELATED PROTEIN 8 |
| APP | AMYLOID BETA A4 PRECURSOR PROTEIN |
| BACE1 | BETA-SITE AMYLOID BETA A4 PRECURSOR PROTEIN-CLEAVING |
| | ENZYME 1 |
| BCHE | BUTYRYLCHOLINESTERASE |
| BDNF | BRAIN-DERIVED NEUROTROPHIC FACTOR |
| BSF1 | INTERLEUKIN 4 |
| CDC2 | CELL DIVISION CYCLE 2, G1 TO S AND G2 TO M |
| CHAT | CHOLINE ACETYLTRANSFERASE |
| CST3 | CYSTATIN 3 |
| CTNNA3 | CATENIN, ALPHA-3 |
| CTSD | CATHEPSIN D |
| CYP2D6 | CYTOCHROME P450, SUBFAMILY IID, POLYPEPTIDE 6 |
| CYP46A1 | CYTOCHROME P450, FAMILY 46, SUBFAMILY A, POLYPEPTIDE 1 |
| DAPK1 | DEATH-ASSOCIATED PROTEIN KINASE 1 |
| DLD | DIHYDROLIPOAMIDE DEHYDROGENASE |
| DLST | DIHYDROLIPOAMIDE S-SUCCINYLTRANSFERASE |
| DNMBP | DYNAMIN-BINDING PROTEIN |
| ESR1 | ESTROGEN RECEPTOR 1 |
| FGF1 | FIBROBLAST GROWTH FACTOR 1 |
| FYN | FYN ONCOGENE RELATED TO SRC, FGR, YES |
| GAPDH | GLYCERALDEHYDE-3-PHOSPHATE DEHYDROGENASE |
| GAPDHS | GLYCERALDEHYDE-3-PHOSPHATE DEHYDROGENASE, |
| | SPERMATOGENIC |
| GSK3B | GLYCOGEN SYNTHASE KINASE 3-BETA |
| GSTO2 | GLUTATHIONE S-TRANSFERASE, OMEGA-2 |
| HHEX | HEMATOPOIETICALLY EXPRESSED HOMEOBOX |
| HLA-A | MAJOR HISTOCOMPATIBILITY COMPLEX, CLASS I, A |
| HLA-DRB1 | MAJOR HISTOCOMPATIBILITY COMPLEX, CLASS II, DR BETA-1 |
| HMGCR | 3-α-HYDROXY-3-METHYLGLUTARYL-CoA REDUCTASE |
| HSPA1A | HEAT-SHOCK 70-KD PROTEIN 1A |
| HSPA1B | HEAT-SHOCK 70-KD PROTEIN 1B |
| HTR2A | 5-α-HYDROXYTRYPTAMINE RECEPTOR 2A |
| HTR6 | 5-α-HYDROXYTRYPTAMINE RECEPTOR 6 |
| ICAM1 | INTERCELLULAR ADHESION MOLECULE 1 |
| IDE | INSULIN-DEGRADING ENZYME |
| IGF1R | INSULIN-LIKE GROWTH FACTOR I RECEPTOR |
| IL18 | INTERLEUKIN 18 |
| IL1A | INTERLEUKIN 1-ALPHA |
| IL1B | INTERLEUKIN 1-BETA |
| IREP2 | IRON-RESPONSIVE ELEMENT-BINDING PROTEIN 2 |
| LDLR | LOW DENSITY LIPOPROTEIN RECEPTOR |
| LPA | APOLIPOPROTEIN(a) |
| LRP1 | LOW DENSITY LIPOPROTEIN RECEPTOR-RELATED PROTEIN 1 |
| LRPAP1 | LOW DENSITY LIPOPROTEIN RECEPTOR-RELATED PROTEIN- |
| | ASSOCIATED PROTEIN 1 |
| LRRK2 | LEUCINE-RICH REPEAT KINASE 2 |
| LTA | LYMPHOTOXIN-ALPHA |
| MAOA | MONOAMINE OXIDASE A |
| MAPK8IP1 | MITOGEN-ACTIVATED PROTEIN KINASE 8-INTERACTING PROTEIN 1 |
| MAPT | MICROTUBULE-ASSOCIATED PROTEIN TAU |
| MME | MEMBRANE METALLOENDOPEPTIDASE |
| MMP1 | MATRIX METALLOPROTEINASE 1 |
| MMP3 | MATRIX METALLOPROTEINASE 3 |
| MMp9 | MATRIX METALLOPROTEINASE 9 |
| MPO | MYELOPEROXIDASE |
| MTHFR | 5,10-α-METHYLENETETRAHYDROFOLATE REDUCTASE |
| NCSTN | NICASTRIN |
| NOS1 | NITRIC OXIDE SYNTHASE 1 |
| NOS3 | NITRIC OXIDE SYNTHASE 3 |
| NOTCH4 | NOTCH, DROSOPHILA, HOMOLOG OF, 4 |
| NP | NUCLEOSIDE PHOSPHORYLASE |
| NQO1 | NAD(P)H DEHYDROGENASE, QUINONE 1 |
| OLR1 | LOW DENSITY LIPOPROTEIN, OXIDIZED, RECEPTOR 1 |
| PARP1 | POLY(ADP-RIBOSE) POLYMERASE 1 |
| PIN1 | PEPTIDYL-PROLYL CIS/TRANS ISOMERASE, NIMA-INTERACTING, 1 |
| PLAU | PLASMINOGEN ACTIVATOR, URINARY |
| PNMT | PHENYLETHANOLAMINE N-METHYLTRANSFERASE |
| PON1 | PARAOXONASE 1 |
| PON2 | PARAOXONASE 2 |
| POU2F1 | POU DOMAIN, CLASS 2, TRANSCRIPTION FACTOR 1 |
| PPARA | PEROXISOME PROLIFERATOR-ACTIVATED RECEPTOR-ALPHA |
| PRNP | PRION PROTEIN |
| PSEN1 | PRESENILIN 1 |
| Psen2 | PRESENILIN 2 |
| PSENEN | PRESENILIN ENHANCER 2, C. ELEGANS, HOMOLOG OF |
| PTGS2 | PROSTAGLANDIN-ENDOPEROXIDE SYNTHASE 2 |
| SNCA | SYNUCLEIN, ALPHA |
| SOAT1 | STEROL O-ACYLTRANSFERASE 1 |
| SOD2 | SUPEROXIDE DISMUTASE 2 |
| SORL1 | SORTILIN-RELATED RECEPTOR |
| STH | SAITOHIN |
| TCN2 | TRANSCOBALAMIN II |
| TF | TRANSFERRIN |
| TFAM | TRANSCRIPTION FACTOR A, MITOCHONDRIAL |
| TFCP2 | TRANSCRIPTION FACTOR CP2 |
| TNFa | TUMOR NECROSIS FACTOR |
| TPH1 | TRYPTOPHAN HYDROXYLASE 1 |
| UBQLN1 | UBIQUILIN 1 |
| UCHL1 | UBIQUITIN CARBOXYL-TERMINAL ESTERASE L1 |
| USF1 | UPSTREAM STIMULATORY FACTOR 1 |
| USF2 | UPSTREAM STIMULATORY FACTOR 2 |
| VLDLR | VERY LOW DENSITY LIPOPROTEIN RECEPTOR |
| WT1 | WT1 GENE |

Complementary to the disease-associated gene markers, disease relevant biomarkers including proteins, polypeptides, enzymes and altered, activated, or phosphorylated forms may be measured. For example, one or more of the Alzheimers markers selected from beta-amyloid 42, tau protein, phosphorylated tau, a-synuclein, BCHE and/or A2M may be measured or assayed. These can be assayed using appropriate blood, urine, or other fluid or cellular samples.

### Diagnostic and Therapeutic Implications

The systems, methods and assays of the present invention have implications in the diagnosis, monitoring and therapeutic intervention of disease, particularly of diseases and conditions which are caused, facilitated, modulated or exacerbated by unresolved oxidative stress or the presence and activity of oxidative stress and its lipid mediators, particularly including metabolites and oxidation products of arachidonic acid (AHA), docosahexanoic acid (DHA) and eicosapentaenoic acid (EPA). Thus, the combination of biochemical and genetic markers can be utilized in a first assessment and determination of oxidative stress and the relevant metabolites in an individual. Continued monitoring, including of the biomarkers and expression of the genetic markers will enable a regular assessment of these parameters in an individual, particularly undergoing therapeutic management. The biomarkers and genetic markers have further use and application in assessing the therapeutic relevant and monitoring a clinical trial or assessment of oxidative stress modulators.

As detailed above, the anti-inflammatory lipid mediators Lipoxins and Resolvins, have been identified and implicated as therapeutic modulators of oxidative stress and stress-related diseases and conditions. Lipoxin compounds and their uses have been reported and described, for instance in U.S. Pat. Nos. 4,560,514, 5,079,261, 5,441,951, 6,635,776, 6,690,674, and 6,887,901, incorporated herein by reference. Anti-inflammatory molecules or compounds derived from EPA, including Resolvin(s), and their uses for diseases, including asthma, gastrointestinal disease, and cardiovascular disease have been described and reported, for instance in U.S. Pat. No. 7,341,840, US20040116408, US20050261255, US20060293288, and US20070254897, incorporated herein by reference. The system and methods of the invention have particular application in trials and assessments of the lipoxins, resolvins and other like molecules and therapies. Characterization and monitoring of stress parameters upon and after administrations of such therapies and molecules provides quantitative and relevant standards for their effects and success.

The continued and regular assessment of the biochemical and genetic markers as detailed herein form an integral and applicable part of the invention. The timing and extent of any such monitoring and assessment, the choice or selection of markers, the methods and samples used can readily be determined by a clinician or one skilled in the art. The collection and interpretation of any such data will readily fall to the skilled artisan using recognized and available methods and approaches.

Genotype may be determined by any means or methods known in the art, including but not limited to genomic Southern blotting, chromosome analysis, sequencing, RNA analysis, expression analysis, and amplification technologies such as PCR.

The nucleic acid assays and methods of the present invention broadly and generally include and incorporate the following steps in determining the genotype of an individual: (a) isolation of nucleic acid from the individual; (b) amplification of relevant nucleic acid or genomic sequence; and (c) analysis of the nucleic acid or genomic sequence. The step (b) may be performed utilizing any method of amplification, including polymerase chain reaction (PCR), ligase chain reaction (Barany, F. (1991) Proc. Natl. Acad. Sci. 88:189-193), rolling circle amplification (Lizardi, P. M. et al (1998) Nature Genetics 19:225-232), strand displacement amplification (Walker, G. T. et al (1992) Proc. Natl. Acad. Sci. 89:392-396) or alternatively any means or method whereby concentration or sequestration of sufficient amounts of the relevant nucleic acid for analysis may be obtained.

In a further embodiment of this invention, commercial test kits suitable for use by a medical specialist may be prepared to determine the biochemical and genetic markers and marker status, and thereby diagnose or monitor a disease or condition associated with unresolved or altered oxidative stress in an individual.

The DNA samples from the persons tested may be obtained from any source including blood, a tissue sample, amniotic fluid, a chorionic villus sampling, cerebrospinal fluid, and urine.

Any of various methods may be used to characterize the relevant genotype of an individual in accordance with the invention. The sequences (nucleic acid and amino acid) of the genes in any of Tables 1, 2, 3, 4, 5 or 6 are known and publically available. Further, one skilled in the art can readily access and/or determine the relevant sequence(s). The skilled artisan can readily design probes, primers, oligonucleotides for determining relevant genotype, and can format or utilize tests or assays based thereon to determine relevant genotype. The tests may utilize PCR, other amplification techniques, allele-specific probes or oligonucleotides, restriction analysis including RFLP analysis, sequencing or such other methods as known or devised. The genotype of the individual, relatives, siblings (affected or unaffected), and/or the fetus can be thus determined by the skilled artisan.

One skilled in the art can use any published, known or recognized method to design primers based on the known sequences of the relevant genes. These primers or probes may be used in methods including PCR methods, SSCP methods, RFLP methods, sequencing methods, allele specific oligonucleotides, etc.

The present invention also provides methods of estimating the genetic susceptibility of an individual to have or to develop a disorder or condition, particularly including autism, asthma and Alzheimer's disease. One such embodiment comprises collecting a biological sample from one or more participants. The participant may be either the individual or a blood relative of the individual. The participant may be a man, woman, child, and/or a pregnant woman. The participants may be a pregnant woman and her fetus. The participants may include the pregnant woman's parents and/or the father of the fetus. The biological sample contains nucleic acids and/or proteins and/or lipids and lipid metabolites of the participant. The nucleic acids and/or proteins and/or lipids and lipid metabolites from the biological sample are analyzed resulting in a determination of biochemical and genetic markers of oxidative stress and a partial or full genotype for the alleles of one or more or several genes associated with or relevant to a specific disease. The combination of markers and a partial or full genotype forms a dataset of relevant markers and alleles for the participant.

Dietary and epidemiological information for environmental explanatory variables for the participant(s) may also be obtained and used to form a dataset of environmental explanatory variables for the participant(s). The datasets of genetic explanatory variables and the dataset of environmental explanatory variables are added to a genetic and environmental reference dataset forming a combined genetic and environmental dataset. A model may be formulated comprising the genetic and environmental explanatory variables obtained from the participant(s). The combined genetic and environmental dataset is then analyzed and a predicted probability for the individual for having and/or developing autism and/or for having offspring that develop autism is determined. The genetic and environmental susceptibility of an individual to have or to develop autism and/or have offspring that develop autism is estimated. Any of known or standard methods for analyzing the combined dataset may be used to determine or assess susceptibility to autism or a related disorder. In an embodiment, analyzing the combined genetic and environmental dataset is performed by binary linear regression. In another embodiment the model is modified by adding or subtracting one or more genetic and/or environmental explanatory variables and the combined genetic and environmental dataset is re-analyzed preferably, by binary logistic regression. A model can then be chosen that best fits the data. This can be accomplished by testing the model for goodness of fit. Exemplary such methods and models are provided and described in U.S. Pat. Nos. 6,210,950 and 6,912,492, which are incorporated herein by reference in their entirety. The skilled artisan can determine the appropriate methods and models, given his knowledge and the statistical and analysis methods known and available.

The invention provides a method for monitoring therapeutic intervention of a disease or condition having unresolved oxidative stress as a component which comprises:

(a) collecting a blood, urine or breath sample for biochemical analysis and isolating nucleic acid from said subject;
(b) analyzing the blood, urine or breath sample to determine selective metabolites and oxidation products of arachidonic acid (AHA), docosahexanoic acid (DHA) and eicosapentaenoic acid (EPA); wherein said analyzing results in a metabolic determination of oxidative stress and lipids; and
(c) analyzing the nucleic acids to determine the genotype and/or expression of genes involved in oxidative stress and/or lipid metabolism;
wherein the existence or severity of a disease or condition is determined.

The invention further contemplates and encompasses kits for the determination and assessment of oxidative stress and concomitant measurement of biochemical and/or genetic markers thereof. The kits thus include the appropriate components to sample and determine selective metabolites and oxidation products of arachidonic acid (AHA), docosahexanoic acid (DHA) and eicosapentaenoic acid (EPA). In a particular such aspect, the components comprise chemically synthesized resolvins and/or lipoxins, including as particularly described herein or as provided by Spur and Rodriguez (Rodriguez A R, Spur B W Tetrahedron Letters (2004) 45 (47): 8717-8720; Rodriguez A R, Spur B W Tetrahedron Letters (2005) 46 (21): 3623-3627; U.S. Patent Ser. No. 60/920,112, filed Mar. 26, 2007, and corresponding PCT filed Mar. 26, 2008, incorporated herein by reference).

It is further contemplated by the present invention to provide methods that include the testing for genetic mutations in individual genes associated with a disease, including autism, asthma and Alzheimer's disease, and/or in individual combinations of such genes. In addition, all possible combinatorials, and permutations of such genes including a constellation comprising all of the genes involved in antioxidant enzymes and oxidative stress is envisioned by the present invention. Alternatively, a constellation of genes in which any one or more genes can be excluded from those tested is also contemplated by the present invention. Thus all of such possible constellations are envisioned by, and are therefore part of the present invention.

Various methods, agents, compounds, and therapies may be used to reduce oxidative stress, and/or act as antioxidants, in the individual. Antioxidant administration, such as high-dose Vitamin C or carnosine may be used (Dolske, M C et al (1993) Prog Neuro-Psychopharmacol Biol Psychiatr 17:765-774; Chez, M G et al (2002) J Child Neurol 17:833-837). Supplementation with betaine and folinic acid or melatonin may be effective. The individual may be treated with glutathione (GSH) or N-acetyl cysteine (NAC). Ubiquinone (coenzyme Q), quercetin, and/or phenolic compounds such as phytoestrogens, flavonoids, and phenolic acid, may have antioxidant effects. Trace elements that are components of antioxidant enzymes such as selenium, copper, zinc, and manganese may be supplemented. Various foods may also act as natural antioxidants such as tomatoes, citrus fruits, carrots, green tea or oolong tea. Other lifestyle changes and stress management techniques may also be implemented. The skilled artisan or medical individual will be familiar with the recognized and emerging modalities/therapies, supplements, compounds, agents which are suitable or applicable for reducing or managing oxidative stress.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Association of Candidate Gene SNPs with Autism

Autism Genetic Resource Exchange (AGRE), a collaborative gene bank for autism, has made available the data from a high density SNP array, the “The Autism Consortium Genome Scan”. This AGRE Affymetrix 5.0 (500K Affy metrix) data was generated at the Broad Institute at MIT and provided to AGRE by Dr. Mark Daly and the Autism Consortium. This scan consisted of 777 families. We gratefully acknowledge the resources provided by the Autism Genetic Resource Exchange (AGRE) Consortium and the participating AGRE families. The Autism Genetic Resource Exchange is a program of Autism Speaks and is supported, in part, by grant 1U24 MH081810 from the National Institute of Mental Health to Clara M. Lajonchere (PI).

We performed a sib-TDT analysis of the AGRE 500K chip for SNPs within our chosen genes using the dfam function of the PLINK package (PLINK v1.00), a tool set for whole-genome associate and population-based linkage analysis (Purcell, S. et al (2007) Am J. Hum Genet 81(3):559-575) We found p=0.00679 and 0.02015 in COX1. We found p=0.01505, 0.06629 and 0.05375 in ALOX12. We found p=0.02516 and 0.08198 in PLA2G6. We found p=0.02682 for a marker about 2000 bp flanking GCLC. We found p=0.06252 and 0.06515 in PLA2G4C. There were no SNPs within the genes COX2 and ALOX5 in this array. No suggestive values were derived in the other genes tested. Values were not corrected for multiple comparisons.

### Example 2

### Correlation of GSTM1*0 Genotype with Isoprostane Excretion in Autism

Because glutathione stransferase M1 (GSTM1) contributes to lowering oxidative stress, the GSTM1*0 allele, a null deletion mutant, is predicted to increase oxidative stress. We have identified GSTM1*0 null deletion as associated with an increased prevalence of autism (Buyske, S. et al BMC Genet (2006) 7:8, and U.S. Patent Application 60/900,573, incorporated herein by reference). Since we previously associated GSTM1*0 with autism it is reasonable to expect that, among individuals with autism, GSTM1*0 homozygotes will have increased oxidative stress compared with non-homozygotes. We have also found increased urinary excretion in autism of an oxidative stress biomarker, isoprostane (Ming, X. et al (2005) Prostaglandins, Leukotrienes and Essential Fatty Acids 73:379-384). Therefore, a reasonable hypothesis is that among individuals with autism, GSTM1*0 homozygotes will excrete larger amounts of isoprostane in their urine than those who are not GSTM1*0 homozygotes. To test this hypothesis, we took advantage of the fact that some autism probands participated in both the GSTM1*0 and the isoprostane studies. We correlated GSTM1*0 homozygosity with urinary isoprostane excretion in these probands in a preliminary study (sample (n=14)) using the Wilcoxon two-sample test and found a significant correlation (p=0.048), supporting this hypothesis. A 1-sided test, although appropriate, was used. A larger study will further assess and validate this correlation.

### REFERENCES

1. Aburatani H, Hippo Y, Ishida T, Takashima R, Matsuba C, Kodama T, Takao M, Yasui A, Yamamoto K, and Asano M. Cloning and characterization of mammalian 8-hydroxyguanine-specific DNA glycosylase/apurinic, apyrimidinic lyase, a functional mutM homologue. Cancer Res 57: 2151-2156, 1997.
2. Akcay T, Saygili I, Andican G, and Yalcin V. Increased formation of 8-hydroxy-2′-deoxyguanosine in peripheral blood leukocytes in bladder cancer. Urol Int 71: 271-274, 2003.
3. Akizawa T, Kinugasa E, and Koshikawa S. Increased risk of malignancy and blood-membrane interactions in uraemic patients. Nephrol Dial Transplant 9 Suppl 2: 162-164, 1994.
4. Ames B N. Endogenous oxidative DNA damage, aging, and cancer. Free Radic Res Commun 7: 121-128, 1989.
5. Amminger G P, Berger G E, Schaefer M R, Klier C, Friedrich M H, and Feucht M. Omega-3 Fatty Acids Supplementation in Children with Autism: A Double-blind Randomized, Placebo-controlled Pilot Study. Biol Psychiatry, in press, 2006.
6. Arita M, Yoshida M, Hong S, Tjonahen E, Glickman J N, Petasis N A, Blumberg R S, and Serhan C N. Resolvin E1, an endogenous lipid mediator derived from omega-3 eicosapentaenoic acid, protects against 2,4,6-trinitrobenzene sulfonic acid-induced colitis. Proceedings of the National Academy of Sciences of the United States of America 102: 7671-7676, 2005.
7. Bazan N G. Cell survival matters: docosahexaenoic acid signaling, neuroprotection and photoreceptors. Trends in Neurosciences 29: 263-271, 2006.
8. Bazan N G, Marcheselli V L, and Cole-Edwards K. Brain response to injury and neurodegeneration: endogenous neuroprotective signaling. Annals of the New York Academy of Sciences 1053: 137-147, 2005.
9. Behrens W A and Madere R. Improved automated method for determining vitamin C in plasma and tissues. Anal Biochem 92: 510-516, 1979.
10. Bier D M. Intrinsically difficult problems: the kinetics of body proteins and amino acids in man. Diabetes-Metabolism Reviews 5: 111-132, 1989.
11. Bieri J G, Tolliver T J, and Catignani G L. Simultaneous determination of alpha-tocopherol and retinol in plasma or red cells by high pressure liquid chromatography. Am J Clin Nutr 32: 2143-2149, 1979.
12. Block G, Dietrich M, Norkus E P, Morrow J D, Hudes M, Caan B, and Packer L. Factors associated with oxidative stress in human populations. Am J Epidemiol 156: 274-285, 2002.
13. Cadet J, Bellon S, Douki T, Frelon S, Gasparutto D, Muller E, Pouget J P, Ravanat J L, Romieu A, and Sauvaigo S. Radiation-induced DNA damage: formation, measurement, and biochemical features. J Environ Pathol Toxicol Oncol 23: 33-43, 2004.
14. Chiang N, Bermudez E A, Ridker P M, Hurwitz S, and Serhan C N. Aspirin triggers antiinflammatory 15-epi-lipoxin A4 and inhibits thromboxane in a randomized human trial. Proceedings of the National Academy of Sciences of the United States of America 101: 15178-15183, 2004.
15. Christie P E, Lee T H, and Spur B W. The effects of lipoxin A(4) on the airway response in asthmatic subjects. Am J Resp Dis 145: 1281-1284, 1992.
16. Clayson D B, Mehta R, and Iverson F. International Commission for Protection Against Environmental Mutagens and Carcinogens. Oxidative DNA damage—the effects of certain genotoxic and operationally non-genotoxic carcinogens. Mutat Res 317: 25-42, 1994.
17. Comstock G W, Norkus E P, Hoffman S C, Xu M W, and Helzlsouer K J. Stability of ascorbic acid, carotenoids, retinol, and tocopherols in plasma stored at −70 degrees C. for 4 years. Cancer Epidemiol Biomarkers Prey 4: 505-507, 1995.
18. Cracowski J L, Devillier P, Durand T, Stanke-Labesque F, and Bessard G. Vascular biology of the isoprostanes. Journal of Vascular Research 38: 93-103, 2001.
19. Dandona P, Thusu K, Cook S, Snyder B, Makowski J, Armstrong D, and Nicotera T. Oxidative damage to DNA in diabetes mellitus. Lancet 347: 444-445, 1996.
20. Davies S S, Zackert W, Luo Y, Cunningham C C, Frisard M, and Roberts L J, 2nd. Quantification of dinor, dihydro metabolites of F2-isoprostanes in urine by liquid chromatography/tandem mass spectrometry. Anal Biochem 348: 185-191, 2006.
21. Ferrante R J, Browne S E, Shinobu L A, Bowling A C, Baik M J, MacGarvey U, Kowall N W, Brown R H, Jr., and Beal M F. Evidence of increased oxidative damage in both sporadic and familial amyotrophic lateral sclerosis. J Neurochem 69: 2064-2074, 1997.
22. Floyd R A. The role of 8-hydroxyguanine in carcinogenesis. Carcinogenesis 11: 1447-1450, 1990.
23. Gangemi S, Luciotti G, D'Urbano E, Mallamace A, Santoro D, Bellinghieri G, Davi G, and Romano M. Physical exercise increases urinary excretion of lipoxin A4 and related compounds. Journal of Applied Physiology 94: 2237-2240, 2003.
24. Helbock H J, Beckman K B, and Ames B N. 8-Hydroxydeoxyguanosine and 8-hydroxyguanine as biomarkers of oxidative DNA damage. Methods Enzymol 300: 156-166, 1999.
25. Hou X, Roberts L J, 2nd, Gobeil F, Jr., Taber D, Kanai K, Abran D, Brault S, Checchin D, Sennlaub F, Lachapelle P, Varma D, and Chemtob S. Isomer-specific contractile effects of a series of synthetic f2-isoprostanes on retinal and cerebral microvasculature. Free Radical Biology & Medicine 36: 163-172, 2004.
26. Hu C W, Wu M T, Chao M R, Pan C H, Wang C J, Swenberg J A, and Wu K Y. Comparison of analyses of urinary 8-hydroxy-2′-deoxyguanosine by isotope-dilution liquid chromatography with electrospray tandem mass spectrometry and by enzyme-linked immunosorbent assay. Rapid Communications in Mass Spectrometry 18: 505-510, 2004.
27. Il'yasova D, Morrow J D, Ivanova A, and Wagenknecht L E. Epidemiological marker for oxidant status: comparison of the ELISA and the gas chromatography/mass spectrometry assay for urine 2,3-dinor-5,6-dihydro-15-F2t-isoprostane. Annals of Epidemiology 14: 793-797, 2004.
28. Kadiiska M B, Gladen B C, Baird D D, Germolec D, Graham L B, Parker C E, Nyska A, Wachsman J T, Ames B N, Basu S, Brot N, Fitzgerald G A, Floyd R A, George M, Heinecke J W, Hatch G E, Hensley K, Lawson J A, Marnett L J, Morrow J D, Murray D M, Plastaras J, Roberts L J, 2nd, Rokach J, Shigenaga M K, Sohal R S, Sun J, Tice R R, Van Thiel D H, Wellner D, Walter P B, Tomer K B, Mason R P, and Barrett J C. Biomarkers of oxidative stress study II: are oxidation products of lipids, proteins, and DNA markers of CCl4 poisoning? Free Radical Biology & Medicine 38: 698-710, 2005.
29. Kadiiska M B, Gladen B C, Baird D D, Graham L B, Parker C E, Ames B N, Basu S, Fitzgerald G A, Lawson J A, Marnett L J, Morrow J D, Murray D M, Plastaras J, Roberts L J, 2nd, Rokach J, Shigenaga M K, Sun J, Walter P B, Tomer K B, Barrett J C, and Mason R P. Biomarkers of oxidative stress study III. Effects of the nonsteroidal anti-inflammatory agents indomethacin and meclofenamic acid on measurements of oxidative products of lipids in CCl4 poisoning. Free Radical Biology & Medicine 38: 711-718, 2005.
30. Kamiya H, Miura K, Ishikawa H, Inoue H, Nishimura S, and Ohtsuka E. c-Ha-ras containing 8-hydroxyguanine at codon 12 induces point mutations at the modified and adjacent positions. Cancer Res 52: 3483-3485, 1992.
31. Kondo S, Toyokuni S, Tanaka T, Hiai H, Onodera H, Kasai H, and Imamura M. Overexpression of the hOGG1 gene and high 8-hydroxy-2′-deoxyguanosine (8-OHdG) lyase activity in human colorectal carcinoma: regulation mechanism of the 8-OHdG level in DNA. Clin Cancer Res 6: 1394-1400, 2000.
32. Lawson J A, Kim S, Powell W S, FitzGerald G A, and Rokach J. Oxidized derivatives of omega-3 fatty acids; Identification of iPF3alpha-VI in human urine. J Lipid Res 47: Web advance publication 2006.
33. Lee C Y, Jenner A M, and Halliwell B. Rapid preparation of human urine and plasma samples for analysis of F2-isoprostanes by gas chromatography-mass spectrometry. Biochemical & Biophysical Research Communications 320: 696-702, 2004.
34. Leinonen J, Lehtimaki T, Toyokuni S, Okada K, Tanaka T, Hiai H, Ochi H, Laippala P, Rantalaiho V, Wirta O, Pasternack A, and Alho H. New biomarker evidence of oxidative DNA damage in patients with non-insulin-dependent diabetes mellitus. FEBS Lett 417: 150-152, 1997.
35. Lin H S, Jenner A M, Ong C N, Huang S H, Whiteman M, and Halliwell B. A high-throughput and sensitive methodology for the quantification of urinary 8-hydroxy-2′-deoxyguanosine: measurement with gas chromatography-mass spectrometry after single solid-phase extraction. Biochemical Journal 380: 541-548, 2004.
36. Lodovici M, Casalini C, Cariaggi R, Michelucci L, and Dolara P. Levels of 8-hydroxydeoxyguanosine as a marker of DNA damage in human leukocytes. Free Radic Biol Med 28: 13-17, 2000.
37. Loft S, Kold-Jensen T, Hjollund N H, Giwercman A, Gyllemborg J, Ernst E, Olsen J, Scheike T, Poulsen H E, and Bonde J P. Oxidative DNA damage in human sperm influences time to pregnancy. Hum Reprod 18: 1265-1272, 2003.
38. Loft S, Larsen P N, Rasmussen A, Fischer-Nielsen A, Bondesen S, Kirkegaard P, Rasmussen L S, Ejlersen E, Tornoe K, Bergholdt R, and et al. Oxidative DNA damage after transplantation of the liver and small intestine in pigs. Transplantation 59: 16-20, 1995.
39. Loft S and Poulsen H E. Cancer risk and oxidative DNA damage in man [published erratum appears in J Mol Med 1997 January; 75(1):67-8]. J Mol Med 74: 297-312, 1996.
40. Loft S and Poulsen H E. Estimation of oxidative DNA damage in man from urinary excretion of repair products. Acta Biochim Pol 45: 133-144, 1998.
41. Loft S and Poulsen H E. Markers of oxidative damage to DNA: antioxidants and molecular damage. Methods Enzymol 300: 166-184, 1999.
42. Loft S, Vistisen K, Ewertz M, Tjonneland A, Overvad K, and Poulsen H E. Oxidative DNA damage estimated by 8-hydroxydeoxyguanosine excretion in humans: influence of smoking, gender and body mass index. Carcinogenesis 13: 2241-2247, 1992.
43. Lu R, Nash H M, and Verdine G L. A mammalian DNA repair enzyme that excises oxidatively damaged guanines maps to a locus frequently lost in lung cancer. Curr Biol 7: 397-407, 1997.
44. Lu Y, Hong S, Gotlinger K, and Serhan C N. Lipid mediator informatics and proteomics in inflammation resolution. Thescientificworldjournal 6: 589-614, 2006.
45. Marczynski B, Kraus T, Rozynek P, Raithel H J, and Baur X. Association between 8-hydroxy-2′-deoxyguanosine levels in DNA of workers highly exposed to asbestos and their clinical data, occupational and non-occupational confounding factors, and cancer. Mutat Res 468: 203-212, 2000.
46. Mecocci P, Polidori M C, Cherubini A, Ingegni T, Mattioli P, Catani M, Rinaldi P, Cecchetti R, Stahl W, Senin U, and Beal M F. Lymphocyte oxidative DNA damage and plasma antioxidants in Alzheimer disease. Arch Neurol 59: 794-798, 2002.
47. Mei N, Tamae K, Kunugita N, Hirano T, and Kasai H. Analysis of 8-hydroxydeoxyguanosine 5′-monophosphate (8-OH-dGMP) as a reliable marker of cellular oxidative DNA damage after gamma-irradiation. Environ Mol Mutagen 41: 332-338, 2003.
48. Miller N J, Rice-Evans C, Davies M J, Gopinathan V, and Milner A. A novel method for measuring antioxidant capacity and its application to monitoring the antioxidant status in premature neonates. Clin Sci (Colch) 84: 407-412, 1993.
49. Ming X, Stein T P, Brimacombe M, Johnson W G, Lambert G H, and Wagner G C. Increased excretion of a lipid peroxidation biomarker in autism. Prostaglandins Leukotrienes & Essential Fatty Acids 73: 379-384, 2005.
50. Montuschi P, Barnes P J, and Roberts L J, 2nd. Isoprostanes: markers and mediators of oxidative stress. FASEB Journal 18: 1791-1800, 2004.
51. Morrow J D. Quantification of isoprostanes as indices of oxidant stress and the risk of atherosclerosis in humans. Arteriosclerosis, Thrombosis & Vascular Biology 25: 279-286, 2005.
52. Morrow J D, Chen Y, Brame C J, Yang J, Sanchez S C, Xu J, Zackert W E, Awad J A, and Roberts L J. The isoprostanes: unique prostaglandin-like products of free-radical-initiated lipid peroxidation. Drug Metab Rev 31: 117-139, 1999.
53. Morrow J D and Roberts L J, 2nd. Mass spectrometric quantification of F2-isoprostanes in biological fluids and tissues as measure of oxidant stress. Methods Enzymol 300: 3-12, 1999.
54. Musiek E S, Cha J K, Yin H, Zackert W E, Terry E S, Porter N A, Montine T J, and Morrow J D. Quantification of F-ring isoprostane-like compounds (F4-neuroprostanes) derived from docosahexaenoic acid in vivo in humans by a stable isotope dilution mass spectrometric assay. Journal of Chromatography B: Analytical Technologies in the Biomedical & Life Sciences 799: 95-102, 2004.
55. Peoples M C and Karnes H T. Recent developments in analytical methodology for 8-hydroxy-2′-deoxyguanosine and related compounds. Journal of Chromatography B: Analytical Technologies in the Biomedical & Life Sciences 827: 5-15, 2005.
56. Plummer S M, Pheasant A E, Johnson R, Faux S P, Chipman J K, and Hulten M A. Evaluation of the relative sensitivity of chromosome painting (FISH) as an indicator of radiation-induced damage in human lymphocytes. Hereditas 121: 139-145, 1994.
57. Pouget J P, Ravanat J L, Douki T, Richard M J, and Cadet J. Measurement of DNA base damage in cells exposed to low doses of gamma-radiation: comparison between the HPLC-EC and comet assays. Int J Radiat Biol 75: 51-58, 1999.
58. Poulsen H E. Oxidative DNA modifications. Experimental & Toxicologic Pathology 57 Suppl 1: 161-169, 2005.
59. Povey A C, Wilson V L, Weston A, Doan V T, Wood M L, Essigmann J M, and Shields P G. Detection of oxidative damage by 32P-postlabelling: 8-hydroxydeoxyguanosine as a marker of exposure. IARC Sci Publ: 105-114, 1993.
60. Pratico D, Rokach J, Lawson J, and FitzGerald G A. F2-isoprostanes as indices of lipid peroxidation in inflammatory diseases. Chemistry & Physics of Lipids 128: 165-171, 2004.
61. Radicella J P, Dherin C, Desmaze C, Fox M S, and Boiteux S. Cloning and characterization of hOGG1, a human homolog of the OGG1 gene of Saccharomyces cerevisiae. Proc Natl Acad Sci USA 94: 8010-8015, 1997.
62. Roberts L J, 2nd, Fessel J P, and Davies S S. The biochemistry of the isoprostane, neuroprostane, and isofuran Pathways of lipid peroxidation. Brain Pathology 15: 143-148, 2005.
63. Roberts L J d, Moore K P, Zackert W E, Oates J A, and Morrow J D. Identification of the major urinary metabolite of the F2-isoprostane 8-iso-prostaglandin F2alpha in humans. J Biol Chem 271: 20617-20620, 1996.
64. Rodriguez A, Nomen M, Spur B W, and Godfroid J J. An efficient asymmetric synthesis of prostaglandin E-1. Eur J Org Chem 2655-2662, 1999.
65. Rodriguez A, Nomen M, Spur B W, Godfroid J J, and Lee T H. Total synthesis of lipoxin A(4) and lipoxin B(4) from butadiene. Tetrahedron Lett 41: 823-826, 2000.
66. Rodriguez A and Spur B W. First toal synthesis of 7(S), 16(R), 17(S) Resolvin D2, a potent anti-inflammatory lipid mediator. Tetrahedron Lett 45: 8717-8720, 2004.
67. Rodriguez A and Spur B W. First toal synthesis of 7(S), 17(S) Resolvin D5, a potent anti-inflammatory docosonoid. Tetrahedron Lett 46: 3623-3627, 2005.
68. Rodriguez A and Spur B W. First total synthesis of E type 1 phytoprostanes. Tetrahedron Lett 44: 7411-7415, 2003.
69. Rodriguez A and Spur B W. Total synthesis of aspirin triggered 15-epi-lipoxin A(4). Tetrahedron Lett 42: 6057-6060, 2001.
70. Rodriguez A and Spur B W. Total synthesis of E-1 and E-2 isoprostanes by diasteroselective protonation. Tetrahedron Lett 43: 9249-9253, 2002.
71. Rodriguez A and Spur B W. Total synthesis of isoprostanes via the two component coupling process. Tetrahedron Lett 43: 4575-4579, 2002.
72. Rokach J, Khanapure S P, Hwang S W, Adiyaman M, Lawson J A, and FitzGerald G A. The isoprostanes: a perspective. Prostaglandins 54: 823-851, 1997.
73. Romano M, Luciotti G, Gangemi S, Marinucci F, Prontera C, D'Urbano E, and Davi G. Urinary excretion of lipoxin A(4) and related compounds: development of new extraction techniques for lipoxins. Laboratory Investigation 82: 1253-1254, 2002.
74. Sastry P S. Lipids of nervous tissue: composition and metabolism. Progress in Lipid Research 24: 69-176, 1985.
75. Scholl T O, Leskiw M, Chen X, Sims M, and Stein T P. Oxidative stress, diet, and the etiology of preeclampsia. American Journal of Clinical Nutrition 81: 1390-1396, 2005.
76. Scholl T O and Stein T P. Oxidant damage to DNA and pregnancy outcome. J Matern Fetal Med 10: 182-185, 2001.
77. Schwarz K B, Kew M, Klein A, Abrams R A, Sitzmann J, Jones L, Sharma S, Britton R S, Di Bisceglie A M, and Groopman J. Increased hepatic oxidative DNA damage in patients with hepatocellular carcinoma. Dig Dis Sci 46: 2173-2178, 2001.
78. Schwedhelm E and Boger R H. Application of gas chromatography-mass spectrometry for analysis of isoprostanes: their role in cardiovascular disease. Clinical Chemistry & Laboratory Medicine 41: 1552-1561, 2003.
79. Serhan C N. Novel eicosanoid and docosanoid mediators: resolvins, docosatrienes, and neuroprotectins. Current Opinion in Clinical Nutrition & Metabolic Care 8: 115-121, 2005.
80. Serhan C N, Gotlinger K, Hong S, Lu Y, Siegelman J, Baer T, Yang R, Colgan S P, and Petasis N A. Anti-inflammatory actions of neuroprotectin D1/protectin D1 and its natural stereoisomers: assignments of dihydroxy-containing docosatrienes. Journal of Immunology 176: 1848-1859, 2006.
81. Shaarawy M, Aref A, Salem M E, and Sheiba M. Radical-scavenging antioxidants in pre-eclampsia and eclampsia. Int J Gynaecol Obstet 60: 123-128, 1998.
82. Shibutani S, Takeshita M, and Grollman A P. Insertion of specific bases during DNA synthesis past the oxidation-damaged base 8-oxodG. Nature 349: 431-434, 1991.
83. Shigenaga M K, Gimeno C J, and Ames B N. Urinary 8-hydroxy-2′-deoxyguanosine as a biological marker of in vivo oxidative DNA damage. Proc Natl Acad Sci USA 86: 9697-9701, 1989.
84. Sperati A, Abeni D D, Tagesson C, Forastiere F, Miceli M, and Axelson O. Exposure to indoor background radiation and urinary concentrations of 8-hydroxydeoxyguanosine, a marker of oxidative DNA damage. Environ Health Perspect 107: 213-215, 1999.
85. Stein T P. Nutrition in the space station era. Nutr Res Rev 14: 87-114, 2001.
86. Stein T P and Leskiw M J. Oxidant damage during and after space flight. Amer J Physiol (Endocrnol and Metab) 278: E375-E382, 2000.
87. Stein T P, Leskiw M J, and Schluter M D. Diet and nitrogen metabolism during spaceflight on the shuttle. J Appl Physiol 81: 82-97, 1996.
88. Stein T P, Schlute M D, Leskiw M J, Chen X, Spur B W, Rodriguez A, and Scholl T O. Oxidative Stress Pathways and Pregnancy Outcome. Proc XV Internation Congress of the Society for Free Radical Research: 43-47, 2006.
89. Suzuki M, Yanagisawa A, and Noyori R. The three component coupling synthesis of prostaglandins. J Am Chem Soc 110: 4718-4726, 1988.
90. Tagesson C, Kallberg M, and Leanderson P. Determination of urinary 8-hydroxydeoxyguanosine by coupled-column high-performance liquid chromotagraphy with electrochemical detection: a noninvasive assay for in vivo oxidative DNA damage in humans. Toxicol Meth 1: 242-251, 1992.
91. Takeuchi T, Nakajima M, and Morimoto K. Establishment of a human system that generates O₂— and induces 8-hydroxydeoxyguanosine, typical of oxidative DNA damage, by a tumor promotor. Cancer Res 54: 5837-5840, 1994.
92. Toraason M, Hayden C, Marlow D, Rinehart R, Mathias P, Werren D, Olsen L D, Neumeister C E, Mathews E S, Cheever K L, Marlow K L, DeBord D G, and Reid T M. DNA strand breaks, oxidative damage, and 1-OH pyrene in roofers with coal-tar pitch dust and/or asphalt fume exposure. Int Arch Occup Environ Health 74: 396-404, 2001.
93. Wako Y, Satoh M, and Suzuki K. 8-Hydroxydeoxyguanosine levels in white blood cell DNA and ex vivo oxidation resistance of plasma in smokers. Tohoku J Exp Med 194: 99-106, 2001.
94. Wilson V L, Taffe B G, Shields P G, Povey A C, and Harris C C. Detection and quantification of 8-hydroxydeoxyguanosine adducts in peripheral blood of people exposed to ionizing radiation. Environ Health Perspect 99: 261-263, 1993.
95. Wood R D, Mitchell M, Sgouros J, and Lindahl T. Human DNA repair genes. Science 291: 1284-1289, 2001.
96. Yao Y, Walsh W J, McGinnis W R, and Pratico D. Altered vascular phenotype in autism: correlation with oxidative stress. Archives of Neurology 63: 1161-1164, 2006.
97. Zhang J, Ichiba M, Hanaoka T, Pan G, Yamano Y, Hara K, Takahashi K, and Tomokuni K. Leukocyte 8-hydroxydeoxyguanosine and aromatic DNA adduct in coke-oven workers with polycyclic aromatic hydrocarbon exposure. Int Arch Occup Environ Health 76: 499-504, 2003.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method for diagnosis or monitoring a disease or condition in an individual comprising:
(a) collecting one or more biological sample from said individual, wherein the biological sample(s) contain proteins, lipids and nucleic acids of the individual;
(b) analyzing the proteins and/or lipids from a biological sample to determine selective metabolites and oxidation products of arachidonic acid (AHA), docosahexanoic acid (DHA) and eicosapentaenoic acid (EPA); wherein said analyzing results in a metabolic determination of oxidative stress and lipids; and
(c) analyzing the nucleic acids from a biological sample to determine the genotype and/or expression of genes involved in oxidative stress and/or lipid metabolism;
wherein the existence or severity of a disease or condition is determined.

2. The method of claim 1 further comprising analyzing the nucleic acids from a biological sample to determine the genotype and/or expression of genes associated with or relevant to a selected disease.

3. The method of claim 1 or 2 wherein analyzing the nucleic acid utilizes PCR analysis.

4. The method of claim 1 or 2 wherein analyzing the proteins or lipids utilizes mass spectrometry.

5. The method of claim 1, wherein step (b) comprises determining levels of one or more of Resolvins D1-D6, E1 or E2 utilizing chemically synthesized and labeled compounds.

6. The method of claim 2, wherein genes associated with a disease selected from autism, asthma, and Alzheimer's disease are analyzed.

7. The method of claim 2 wherein the disease is autism and the genotype and/or expression of one or more genes set out in Table 4 are determined.

8. The method of claim 2 wherein the disease is asthma and the genotype and/or expression of one or more genes set out in Table 5 are determined.

9. The method of claim 2 wherein the disease is Alzheimer's disease and the genotype and/or expression of one or more genes set out in Table 6 are determined.

10. An assay system for diagnosis or monitoring a disease or condition having unresolved oxidative stress as a component which comprises:
(a) collecting a blood, urine or breath sample for biochemical analysis and isolating nucleic acid from said subject;
(b) analyzing the blood, urine or breath sample to determine selective metabolites and oxidation products of arachidonic acid (AHA), docosahexanoic acid (DHA) and eicosapentaenoic acid (EPA); wherein said analyzing results in a metabolic determination of oxidative stress and lipids; and
(c) analyzing the nucleic acids to determine the genotype and/or expression of genes involved in oxidative stress and/or lipid metabolism;
wherein the existence or severity of a disease or condition is determined.

11. A method for monitoring therapeutic intervention of a disease or condition having unresolved oxidative stress as a component which comprises:
(a) collecting a blood, urine or breath sample for biochemical analysis and isolating nucleic acid from said subject;
(b) analyzing the blood, urine or breath sample to determine selective metabolites and oxidation products of arachidonic acid (AHA), docosahexanoic acid (DHA) and eicosapentaenoic acid (EPA); wherein said analyzing results in a metabolic determination of oxidative stress and lipids; and
(c) analyzing the nucleic acids to determine the genotype and/or expression of genes involved in oxidative stress and/or lipid metabolism;
wherein the existence or severity of a disease or condition is determined.
